(19) United States
(12) Patent Application Publication
(10) Pub. No.: US 2020/0206198 A1
(43) Pub. Date: Jul. 2, 2020

(54) CFTR CHANNEL ACTIVATOR FOR USE IN THE TREATMENT AND/OR PREVENTION OF GLUTEN SENSITIVITY CONDITIONS

(71) Applicant: Luigi Maiuri, Napoli (NA) (IT)

(72) Inventor: Luigi Maiuri, Napoli (NA) (IT)

(21) Appl. No.: 16/633,582

(22) PCT Filed: Jul. 25, 2018
(86) PCT No.: PCT/EP2018/070103

§ 371 Date: Jan. 24, 2020

(30) Foreign Application Priority Data
Jul. 26, 2017 (IT) 102017000085714

(51) Int. Cl. A61K 31/4245; A61K 9/00

(52) U.S. Cl. CPC A61K 31/4245; A61K 9/0053

(57) ABSTRACT

The present invention relates to a CFTR channel activator, or a pharmaceutical composition thereof, for use in the treatment and/or prevention of conditions of gluten sensitivity, such as a) celiac disease (CD) and/or a celiac-associated condition and/or gluten-related diseases, selected from potential celiac disease, refractory celiac disease, type 1 diabetes, autoimmune thyroiditis, or (b) non-celiac gluten sensitivity (NCGS) or irritable bowel disease.

The present invention relates to the use of a CFTR channel activator, or a pharmaceutical composition thereof, in the treatment and/or prevention of conditions of gluten sensitivity, such as celiac disease (CD) and celiac-associated conditions, gluten-related diseases and non-celiac gluten-sensitive (NCGS) disorders.

The intestinal immune system is confronted with the permanent challenge to distinguish between safe and potentially harmful luminal triggers. Under physiological conditions, a finely tuned system of cellular adaptation ensures tissue homeostasis and provides the gut mucosa with the unique capacity of suppressing inflammation and promoting oral tolerance to non-self antigens from dietary origin or commensal microbes. This tolerogenic response can be subverted by environmental triggers, such as viral infections (Bouziat, R., et al. 2017), or as yet undefined predisposing factors, leading to immune and inflammatory response.

Celiac disease (CD) is most frequent permanent intolerance to dietary proteins on an immunological basis occurring in 1% of individuals worldwide. In a subset of genetically susceptible individuals expressing human leukocyte antigen (HLA) DQ2/DQ8 molecules, the ingestion of gluten proteins from wheat, barley and rye and related cereal proteins switches the physiological tolerogenic behavior of the intestinal mucosa toward an adaptive immune response with an autoimmune component characterized by the production of autoantibodies against the self antigen Tissue transglutaminase (TG2). In CD intestine, gluten proteins trigger an HLA DQ2/8-restricted T helper 1 (TH1) immune response. Gluten-specific CD4+ T cells work in concert with intestinal B cells to induce villus damage and production of IgA antibodies against the self-antigen tissue transglutaminase (TG2) (Sollid, L. M. et al. 2011), resulting in an autoimmune response (Meresse, B. et al. 2012; Sollid, L. M et al. 2013). Indeed, besides the multifaceted clinical manifestations of CD, including either gastrointestinal and extraintestinal symptoms, in celiac individuals there is an increased risk to develop other autoimmune disorders as exemplified by the association with type 1 diabetes or autoimmune thyroiditis.

However, the adaptive immune response against gluten is not sufficient, albeit necessary, to trigger overt CD. Additional genetic or environmental predisposing factors are required to induce epithelial stress response with innate immune activation, essential for cytotoxic activation of CD8+T intraepithelial lymphocytes which are indispensable for triggering villus atrophy. Recently, reovirus infections or other yet to be defined environmental factors have been proposed as putative triggers (Bouziat, R., et al. 2017).

Gliadin, a protein fraction from gluten, contains amino acid sequences that are capable of triggering epithelial/innate immunity activation on their own. Some peptide fractions encompassing the α-gliadin amino acid sequence LGQQQPFPPQQPY (P31-43) or an extended sequence (P31-49), are known to induce stress response in intestinal epithelial cell lines and to trigger innate immunity activation in celiac biopsy cultures (Maiuri, L., et al. 2003; Meresse, B. et al.2009; Barone, M. V. et al. 2014). Such gliadin sequences can induce in celiac duodenal biopsies the mucosal stress required to enable immunodominant gliadin epitopes to elicit a Th1 immune response. Thus, gliadin peptides curb major mechanisms of cell/tissue adaptation to stressing conditions through yet unknown mechanisms.

The only recognized treatment of CD patients is the gluten-free diet. However, GFD can generate psychological problems in CD patients who often show a poor compliance to the diet. Thus, a novel approach which allow CD patients to peacefully coexist with gluten is needed.

Several experimental treatments are at the forefront of research in CD, aiming at either gluten manipulation prior ingestion by enzymatic treatments (Eugster, P. J. et al. 2015) or modifying the downstream immunological host response (Goel, G. 30 et al 2017). However, an appropriate treatment for celiac individuals is far to be provided in clinical practice. Thus, at the present, a strict gluten-free diet is the only treatment available for celiac individuals who need lifelong gluten exclusion to avoid disease manifestations and prevent the occurrence of celiac-related conditions, as autoimmune (Meresse, B. et al. 2012; Sollid, L. M et al. 2013) or neoplastic diseases. In addition, a growing proportion of non-celiac individuals who exhibit gastrointestinal or extra-intestinal clinical symptoms upon gluten consumption can take advantage from a gluten-free diet with clear improvement of their clinical status.

In view of the above, the Applicant has faced the problem of providing a new therapeutic approach to intestinal or extra-intestinal conditions of gluten sensitivity selected from celiac disease (CD), celiac associated conditions and non-celiac gluten sensitivity (NCGS).

To that purpose, the Applicant has decided to focus on c(AMP)-regulated Cystic Fibrosis Transmembrane Conductance Regulator (CFTR).

In fact, using different in-vitro, ex vivo and in-vivo pre-clinical mouse and human experimental models of gluten sensitivity, it was surprisingly found that the effects of gliadin on the small intestine are mediated by its ability to reduce at the intestinal surface the function of CFTR.

In addition, the Applicant has found that maintaining the CFTR channel in a functional open state by means of activators of CFTR channel activity, the intestinal mucosa can be protected from gluten-driven mucosal stress and immunity activation and restores oral tolerance to gluten.

Therefore, the Applicant has found that through a CFTR channel activator it is possible to treat and/or prevent conditions of gluten sensitivity. In particular, through a CFTR channel activator it is possible to treat and/or prevent conditions of gluten sensitivity, such as celiac disease (CD) and celiac-associated conditions, gluten-related diseases and non-celiac gluten-sensitive (NCGS).

Therefore, according to a first aspect, the invention relates to a CFTR channel activator for use in the treatment and/or prevention of at least one intestinal and/or extra-intestinal condition of gluten sensitivity selected from:

a) celiac disease (CD) and/or a celiac-associated condition and/or gluten-related diseases, selected from potential celiac disease, refractory celiac disease, type 1 diabetes, autoimmune thyroiditis, or (b) non-celiac gluten sensitivity (NCGS).

Advantageously, the use of a CFTR channel activator allows to provide a valid therapeutic and/or prevention method with reference to intestinal and/or extra-intestinal conditions of gluten sensitivity.

Particularly, the use of a CFTR channel activator represents an alternative to the mere gluten removal from the diet, as commonly occurs, for example, for CD. Indeed, no etiological therapy exists for individuals with non-celiac gluten sensitivity who suffer from a wide range of gastrointestinal or extra-intestinal, even systemic, symptoms that are currently treated with symptomatic therapies. A growing proportion of such patients can benefit from a gluten-free diet resulting in improvement of clinical manifestations.

Further characteristics and advantages of the invention will be evident from the following detailed description.

### DETAILED DESCRIPTION

For the scopes of the invention, the terms and expressions reported below are to be intended as follows, unless otherwise specified.

The term “CFTR channel activator” of the invention includes any potentiator, or amplifier capable of modulating the CFTR channel function.

The term “CFTR potentiator” as used herein means a compound capable of enhancing CFTR channel function.

The term “CFTR amplifier” as used herein means a compound capable of increasing the PM expression of the CFTR protein.

According to a preferred embodiment of the invention, the CFTR channel activator is a CFTR potentiator. Preferably, said CFTR potentiator is selected from N-(2,4-di-tert-butyl-5-hydroxyphenyl)-4-oxo-1,4-dihydroquinoline-3-carboxamide (VX-770, Ivacaftor), 2-[(2-1H-indol-3yl-acetyl)-methyl- amino]-N-(4-isopropyl-phenyl)-2phenyl-acetamide (PG01,P2), 4-Methyl-2-(5-phenyl-1Hpyrazol-3-yl)-phenol (P1, VX-532), 6-(Ethyl-phenyl-sulfonyl)-4-oxo-1,4-dihydro-quinoline-3-carboxylic acid 2-methoxy-benzylamide (SF-03, P3), 1-(3-chlorophenyl)-5-trifluoromethyl-3-hydrobenzimidazol2-one (UCCF-853, P4), 2-(2-Chloro-benzoylamino)4,5,6,7-tetrahydrobenzo[b]thiophene-3-carboxylic acid amide (P5), 5,7,Dihydroxy-3-(4-hydroxy-phenyl)-chroman-4-one, 145-Chloro-2-hydroxy-phenyl)-5-trifluoromethyl-1,3-dihydro-indol2-one (NS004, P7), 4-(4-Oxo-4H-benzo[h]chromen-2yl)-pyridinium; bisulfate (P8), 3-But-3-ynyl-5-methoxy-1-phenyl-1H-pyrazole-4-carb aldehyde (P9), 3-(2-Benzyloxy-phenyl)-5-chloromethyl-isoxazole (P10), P5 analogues P11, P12, P13, P14, P15, P16, P17, P18, P19, P20, 2-thioxo-4-amino-thiazoles (A01 and A02), A01 analogues as A03, A04, A05, A06, A07, A08, A09, pyrazole-pyrrole isoxazoles as H01, H02, H03, H04, H05, H06, H07, H08, H09, H10, GLPG1837, GLPG2451, GLP3067, Q8W251, FDL169, C-10355, C-10358, PTI-808, Aminoarylthiazoles (AATs),tetrahydrobenzothiophenesthioxoaminothiazoles, pyrazole-pyrrole-isoxazole, 5-Nitro-2-(3-Phenylpropylamino Benzoate, 4,6,4′-trimethylangelicin, with the proviso that said potentiator does not include quercitin, curcumin, silybin, apigenin or antocyanidins.

Preferably, the CFTR potentiator is selected from N-(2,4-di-tert-butyl-5-hydroxyphenyl)-4-oxo-1,4dihydroquinoline-3-carboxamide (VX-770, Ivacaftor), 5,7-Dihydroxy-3-(4-hydroxy-phenyl)-chroman-4-one, 4-Methyl-2-(5-phenyl-1H-pyrazol-3-yl)-phenol or mixtures thereof.

According to a preferred embodiment of the invention, the CFTR channel activator is a CFTR amplifier. Preferably, said CFTR amplifier is PTI-428 (Proteostasis Therapeutics Inc-428 by the Company: Proteostasis Therapeutics Inc.).

It will be understood that any form of the CFTR channel activator as defined above, (i.e. free base, pharmaceutically acceptable salt, solvate, etc.) that is suitable for the particular mode of administration can be used in the pharmaceutical compositions discussed herein.

In a preferred embodiment, the invention provides the administration of a combination of two or more CFTR channel activators. Preferably the use of such combination is a simultaneous, separate or sequential use. Particularly, “simultaneous use” is understood as meaning the administration of the at least two CFTR activators according to the invention in a single pharmaceutical form. “Separate use” is understood as meaning the administration, at the same time, of the at least two CFTR activators according to the invention in distinct pharmaceutical forms. “Sequential use” is understood as meaning the successive administration of the at least two CFTR activators according to the invention, each in a distinct pharmaceutical form.

According to a further aspect, the invention relates to a pharmaceutical composition comprising a CFTR channel activator as defined above and at least one pharmaceutically acceptable excipient for use in the treatment and/or prevention of at least one intestinal and/or extra-intestinal condition of gluten sensitivity selected from:

(a) celiac disease (CD) and/or a celiac-associated condition and/or gluten-related diseases, selected from potential celiac disease, refractory celiac disease, type 1 diabetes, autoimmune thyroiditis, or

(b) non-celiac gluten sensitivity (NCGS).

The term “excipient” as used herein describes a material that does not cause significant irritation to an organism and does not abrogate the biological activity and properties of the active principle of the composition according to the invention. Excipients must be of sufficiently high purity and of sufficiently low toxicity to render them suitable for administration to a subject being treated. The excipient can be inert, or it can possess pharmaceutical benefits.

According to a preferred aspect of the invention, the CFTR channel activator or the composition as defined above may be administered to the subject by any acceptable route of administration including, but not limited to, inhaled, oral, nasal, topical (including transdermal and rectal) and parenteral modes of administration. A particularly preferred composition is a small intestine-specific drug oral administration form. Examples of said small intestine-specific compositions include tablets, capsules, hydrogels and the like, characterized by excipients such as pH sensitive polymers allowing a selective release of the active ingredient in the small intestine.

Further, according to another preferred aspect of the invention, the CFTR channel activator or the composition as defined above may be administered in multiple doses per day, in a single daily dose or a single weekly dose. It will be understood that any form of the active agents used in the composition of the invention, (i.e. free base, pharmaceutically acceptable salt, solvate, etc.) that is suitable for the particular mode of administration can be used in the pharmaceutical compositions discussed herein.

The invention will now be further illustrated by means of the following examples.

### Example 1

The α-Gliadin-Derived Peptide P31-43 Inhibits CFTR Function in Intestinal Epithelial 5 Cells

Human colon adenocarcinoma-derived Caco-2 and T84 cells (Barone, M. V. et al. 2014; Luciani, A. et al. 2010) were obtained from the ATCC. Cells were maintained in T25 flask in Modified Eagle Medium (MEM) for Caco-2, or Ham's F12+DMEM (1:1) for T84, supplemented with 10% fetal bovine serum (FBS), 2 mM Glutamine+1% Non Essential Amino Acids (NEAA) and the antibiotics penicillin\streptomycin (100 units/ml) (all reagents from Lonza). Caco-2 cells were grown in Transwells (Corning, 3470 or 3460) under the normal condition. Briefly, 8×10⁴ or 5×10⁵ cells were seeded in 6.5-mm diameter or 12-mm diameter, respectively, and grown until the RT reached 800 to 1,200 Ω·cm². Transwells with a pore size of 0.4 μm were used. Medium in both the apical and baso lateral chambers was changed every other day. Cells were treated with 20 μg/ml of either with the α-gliadin peptide comprising the P31-43 amino acid sequence of α-gliadin LGQQQPFPPQQPY (SEQ ID 1) (below referred to as P31-43), which is known to induce an enterocyte stress response (Meresse, B. et al.2009; Barone, M. V. et al. 2014; Luciani, A. et al. 2010), the T-cell activating α-gliadin peptide comprising the P57-68 amino acid sequence of α-gliadin (QLQPFPQPQLPY SEQ ID 2)) (below referred to as P57-68) (Maiuri, L., et al. 2003; Meresse, B. et al.2009; 30 Barone, M. V. et al. 2014), or the scrambled GAVAAVGVVAGA (SEQ ID 3) control peptide (below referred to as PGAV), or with different modified P31-43, either biotin-tagged or not, for different time points (from 1 h short challenge up to 24 h). Cells were also treated with 50 or 100 5 μg/ml of P57-68 or PGAV peptides for 3 h.

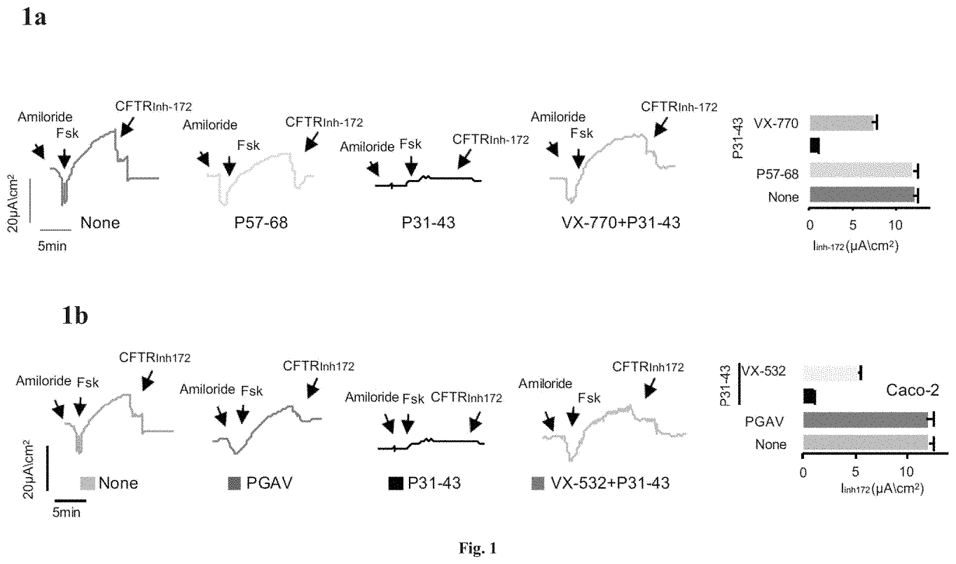

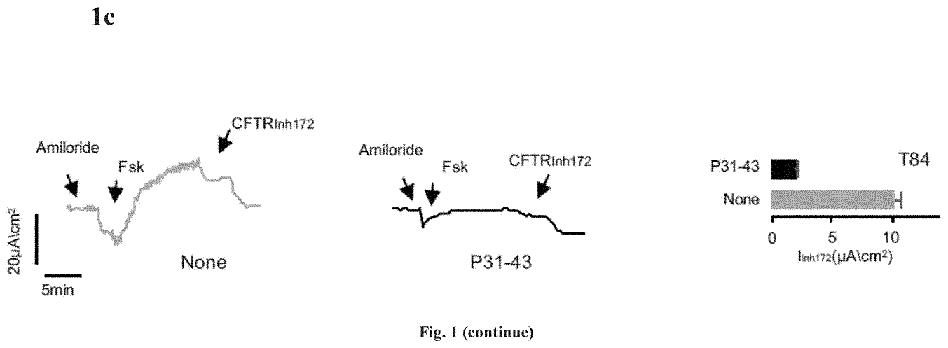

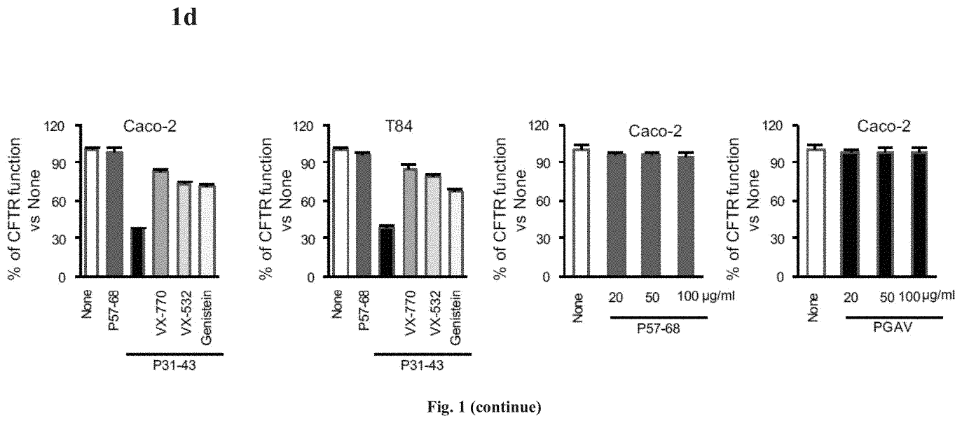

The Applicant found that 20 μg/ml of P31-43, but neither P57-68 nor PGAV even if tested at 50 or 100 μg/ml, did reduce CFTR channel function in response to a short pulse of the cAMP activator forskolin (FIG. 1).

### Example 2

CFTR Potentiators Ivacaftor, VX-532 or Genistein Prevents P31-43 Induced Inhibition of CFTR Function in Intestinal Epithelial Cells.

Caco-2 or T84 cells were also treated with: CFTR potentiators VX-770 (Ivacaftor) (10 μM) or VX-532 (20 μM) (Selleck chemicals) or Genistein (10 μM) (Verkman, A. S. et al. 2005; Jih, K. Y. et al. 2013) (Sigma-Aldrich) or in presence or absence of CFTR inhibitor172 (CFTRᵢₙₕ₁₇₂, 20 μM). The Applicant found that all tested CFTR potentiators prevented the decrease of CFTR function induced by P31-43 (FIG. 1).

### Example 3

P31-43 Binds to NBD1 Domain of CFTR.

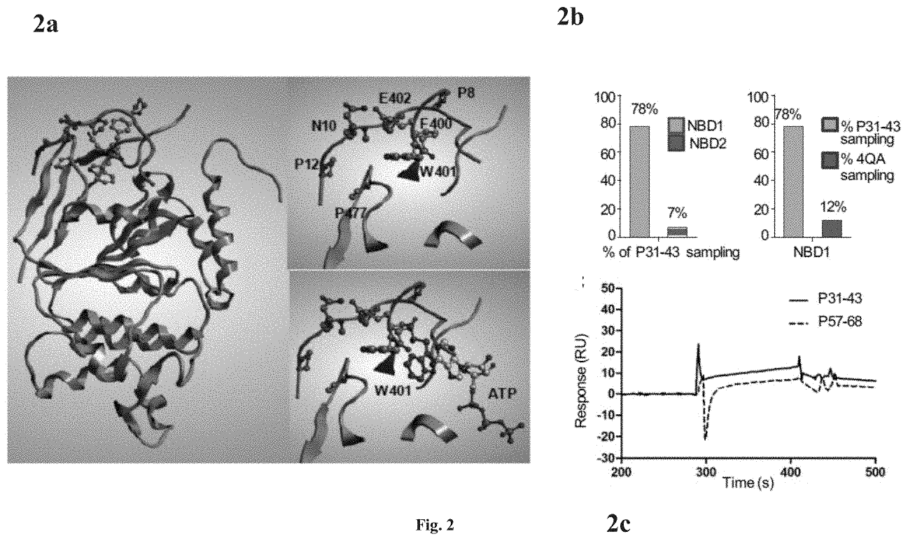

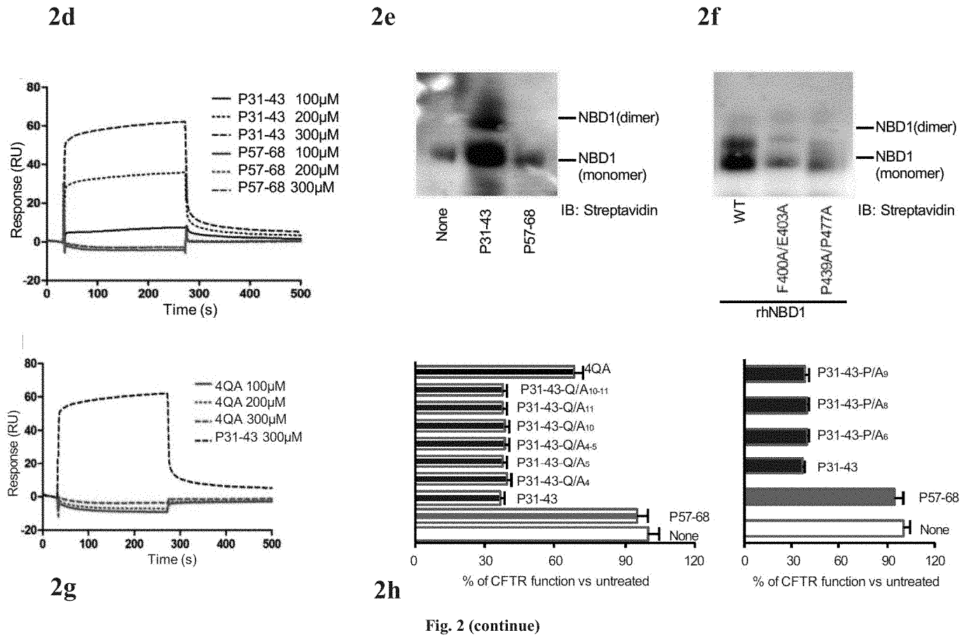

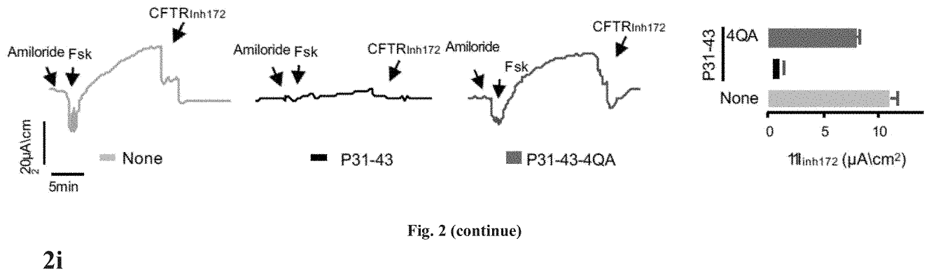

To unravel how P31-43 may impair CFTR channel activity, the Applicant investigated whether P31-43 may target the nuclear binding domains (NBDs) of CFTR, which are responsible for ATP binding and hydrolysis (Sheppard, D. N et al. 1999). Protein-protein docking analysis suggested a high probability (78%) for P31-43 interaction with NBD1 (between amino acids 400 and 477) (FIG. 2a,b) but a low probability (7%) for its interaction with NBD2 (FIG. 1c). Molecular dynamics simulation indicated that P31-43 forces the movement of tryptophan at the position 401 (W401), which normally interacts with the adenine moiety of ATP via π-π stacking, from its original position to a novel one that is not any more compatible with the interaction with ATP and hence with ATPase activity (FIG. 2a). Surface plasmon resonance (SPR) confirmed the predicted interaction of P31-43 (but not P57-68) immobilized on a CMS sensor chip with recombinant human NBD1(rhNBD1) in the flow phase (FIG. 2c). This selective interaction was also detected by SPR when rhNBD1 was immobilized on the chip and exposed to different concentrations of either P31-43 (which yielded a dose-dependent signal) and P57-68 (which failed to induce any signal)(FIG. 2d). Molecular modelling indicated that, within NBD1, the amino acid couples F400/E403 and P439/P477 are critical for the interaction with P31-43 (FIG. 2a). This model could be confirmed by incubating native rhNBD1 or its two double mutants F400A/E403A and P439A/P477A with P31-43 and subjecting the complex to native polyacrylamide electrophoresis. P31-43 (but not P5768) bound to rhNBD1 (FIG. 2e), and this interaction was attenuated for both rhNBD1 mutants (FIG. 20.

The in silico model (FIG. 2a) also led to the prediction that substitution of the glutamines at residues 4, 5, 10 and 11 with alanines (LGQAAPFPPAAPY-SEQ ID 4), below referred to as 4QA) would attenuate binding to NBD1 (FIG. 2b). Indeed, the models predicts that Q4 and Q10 within the gliadin peptide P31-P43 directly interact with NBD1 residues E403 and E402 respectively, while Q5 and Q10 establish intrapeptide interactions with P5 and P12 that are likely essential for their correct orientation and binding to NBD1 residues P439/F400 and P477, respectively. Notably, the interaction of P31-43 with E402 likely accounts for the forced movement of W401 described above (FIG. 2c). In accord with the in silico predictions, SRP revealed that the mutant peptide 4QA failed to interact with NBD1 (FIG. 2g).

In line with the in silico prediction and cell-free experiments discussed above, the quadruple mutated P31-43 peptide 4QA, failed to interact with CFTR and to inhibit its function in intestinal epithelial cells, contrasting with single or double Q/A substitutions in P31-43 that did not affect the CFTR-inhibitory effect of P31-43 (FIG. 2h,i).

### Example 4

P31-43 Reduces NBD1 ATPase Activity

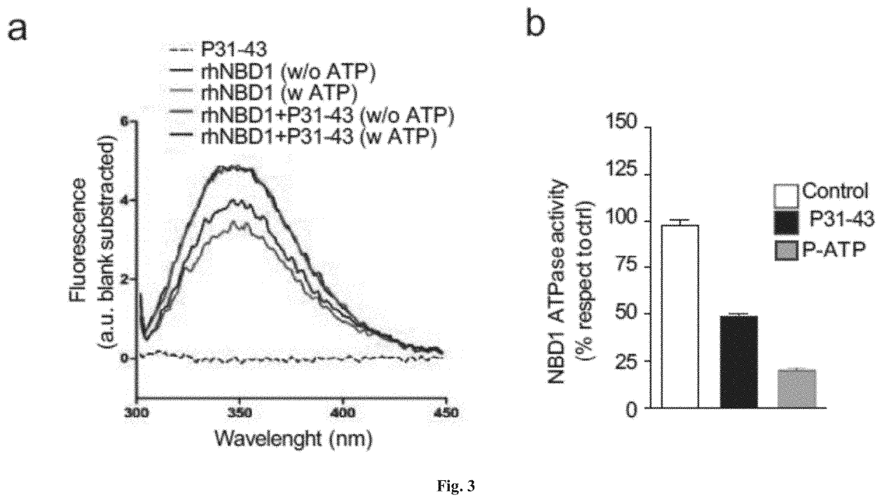

Next, the Applicant investigated whether the interaction between P31-43 and NBD1 interferes with ATP binding as this was predicted from the altered spatial orientation of W401. When measuring the intrinsic tryptophan fluorescence, the Applicant observed that the quenching effect of ATP on W401 fluorescence was reduced by preincubation of rhNBD1 with P31-43 (FIG. 3a). Notably, the data shown in FIG. 1i suggest that P3143 does not enter the ATP binding pocket, thus confirming the in silico hypothesis that a conformational change occurs indirectly when P31-43 binds NBD1. Next, the Applicant assessed the effects of P31-43 on the ATPase capacity of NBD1 in the presence of P31-43 or of a non-hydrolyzable ATP analogue (P-ATP) as a positive control of inhibition. P31-43 inhibited NBD1 ATPase activity with an IC50 of ˜50 μM (FIG. 3b). These experiments confirm the in silico prediction and indicate that the direct binding of P31-43 to NBD1 negatively affects the ATPase activity of NBD1 via a conformational effect.

P31-43 Interacts with CFTR in Epithelial Cells

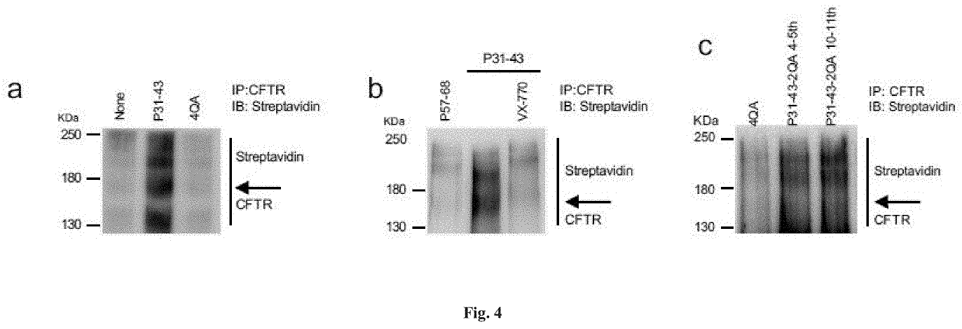

Next, the Applicant incubated intestinal epithelial (Caco-2) cells for 1 h with biotinylated peptides and demonstrated that P31-43, but not P57-68, co-immunoprecipitated with CFTR shortly upon challenge (FIG. 4a-b). The mutated P31-43 (Q4A) did not co-immunoprecipitate with CFTR (FIG. 4c). VX-770 prevented the co-immunoprecipitation between P31-43 and CFTR (FIG. 4b).

### Example 5

CFTR Potentiators Prevent P31-43 Induced Epithelial Stress

Next, the Applicant determined whether CFTR inhibition may account for the P31-43-induced epithelial stress response, which is pivotal for CD pathogenesis. (Cerf-Bensussan, N. et al. 2015; Meresse, B. et al.2009; Barone, M. V. et al. 2014). Indeed, disabling the CFTR can generate oxidative stress, increase intracellular Ca2+ concentrations and Ca2+-dependent activation of TG2 (Maiuri, L., et al. 2008; Luciani, A., et al. 2009), causing major perturbations of proteostasis (Villella, V. R., et al. 2013; Luciani, A. et al. 2010).

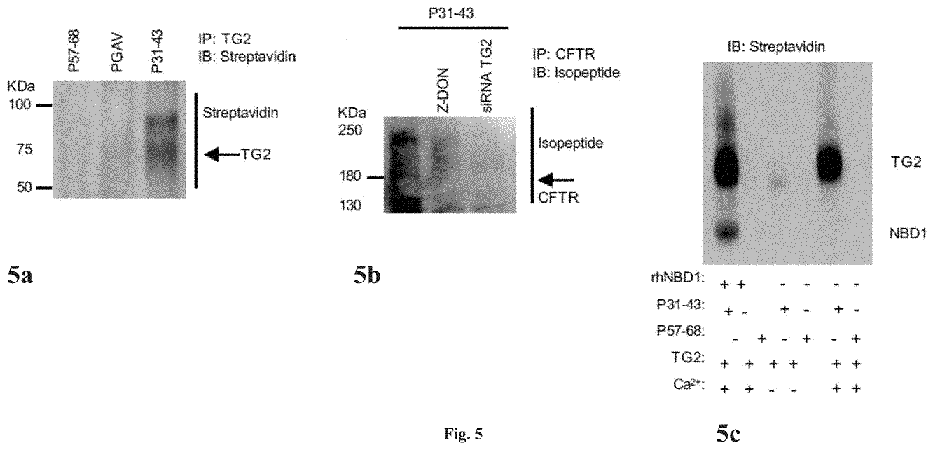

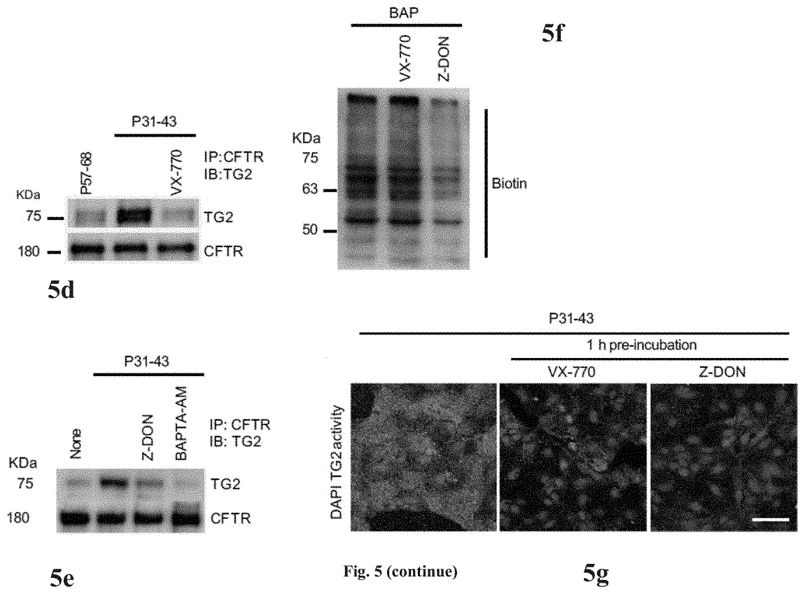

Consistent with the ability of P31-43 to activate TG2 and to be transamidated by TG2 (Siegel, M. et al. 2007; Meresse, B. et al.2009; Barone, M. V. et al. 2014), P31-43 (but not P57-68) covalently interacted with TG2 in Caco-2 cells shortly upon challenge (FIG. 5a). P31-43 also induced the formation of TG2-mediated glutamime-lysine bonds that were detectable in CFTR immunoprecipitates (FIG. 5b). Notably, in a cell-free system containing recombinant proteins and synthetic peptides, Ca2+-activated TG2 catalyzed covalent interactions between P31-43 and NBD1 (FIG. 5c).

In addition, the incubation of cells with P3143 stimulated the interaction between TG2 and CFTR detectable by co-immunoprecipitation (FIG. 5d), an effect prevented by the Ca2+chelator BAPTA-AM, as well as by the inhibition of TG2 transamidating activity with Z-DON (Siegel, M. et al. 2007; Rauhavirta, T., et al. 2013) (FIG. 5e).

Notably, the Applicant discovered that the preincubation of Caco-2 cells with the CFTR potentiator VX-770, which has no impact on TG2mediated transamidation reactions (FIG. 5f), abolished the P31-43-induced interaction of TG2 and CFTR (FIG. 5d) and negated the capability of P31-43 5 to induce TG2 activation in Caco-2 cells (FIG. 5g).

P31-43 also stimulated TG2-mediated cross-linking of the regulatory subunit 2a of Protein Kinase A (PKAr2a) (FIG. 5h), leading to a decrease in the abundance of phosphorylated PKA protein (FIG. 5i). Since PKA is essential for CFTR phosphorylation and activity (Chin, S. et al. 2017) this effect may further compromise CFTR function. Importantly, VX-770 counteracted all these TG2-mediated effects of P31-43 (FIG. 5h,i). Moreover, VX-770 protected Caco-2 cells from other signs of P31-43-induced epithelial stress including the downregulation of PPARγ (FIG. 5j, top), the phosphorylation of ERK-1/2 (Barone, M. V. et al. 2014; Luciani, A. et al. 2010) (FIG. 5j, bottom) and the upregulation of heat shock protein (HSP) 70 expression (FIG. 5k).

Thus, P31-43 binding to CFTR impairs CFTR channel function, thus inducing TG2 activation that in turn stabilizes P31-43/CFTR interaction and sustains P31-43 induced epithelial stress response. Counteracting P31-43 binding and subsequent CFTR hypofunctioning through VX-770 abrogates P31-43 toxic activity on intestinal epithelial cells.

### Example 6

CFTR Potentiators Protect Epithelial Cells from P31-43 Induced Alteration of Endosomal Trafficking

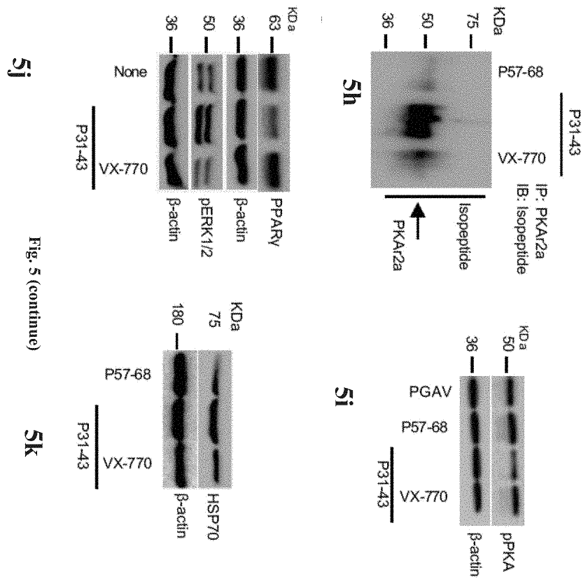

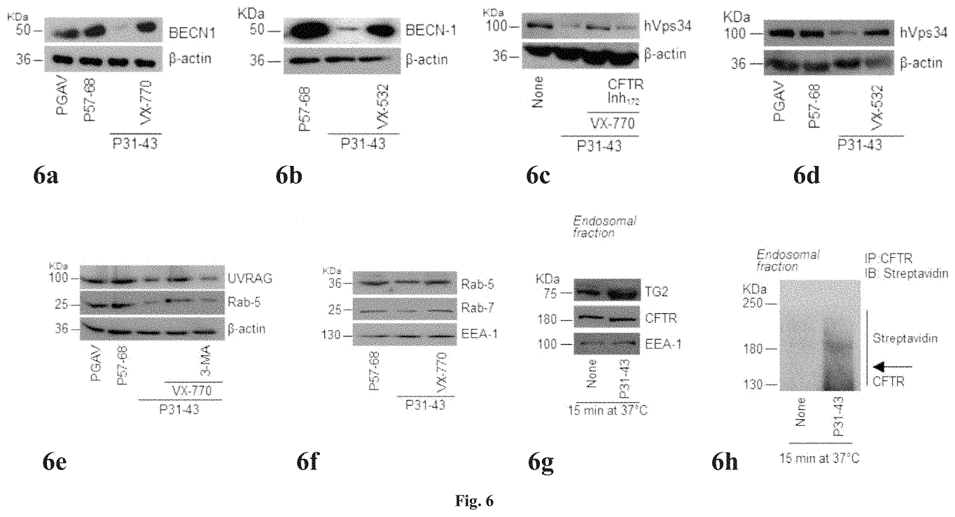

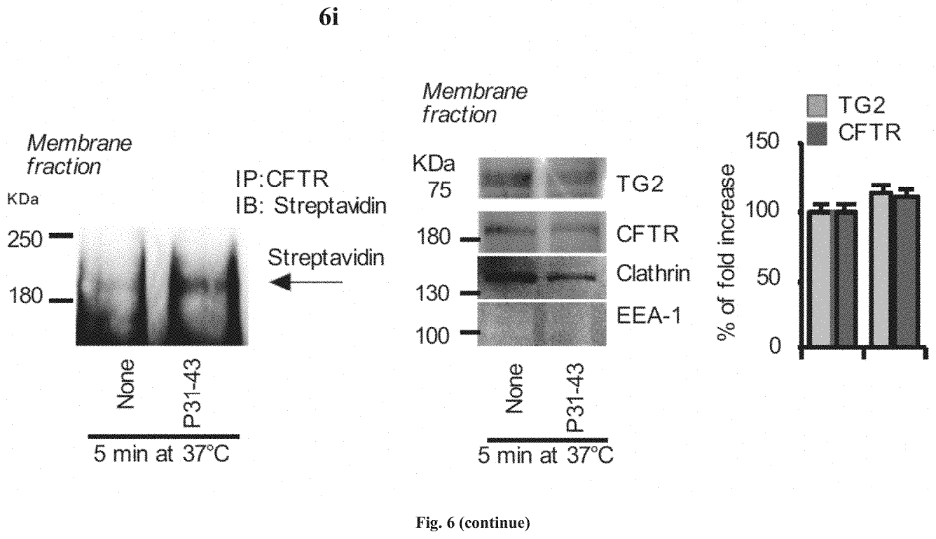

A major consequence of CFTR malfunction in epithelial cells is the impairment of endosomal maturation and trafficking (Villella, V. R., et al. 2013) consequent to TG2-mediated sequestration of the phosphatidyl-inositol-3-kinase (PI3K) (Villella, V. R., et al. 2013; Luciani, A. et al. 2010) complex-3 organized around the Beclin 1 (BECN1) protein and its major interactors PI3K (also named hVps34), which is essential for autophagosome formation, and UV-irradiation-resistant-associated-gene (UVRAG), which is pivotal for endosomal maturation and trafficking. P31-43, as well as the α-gliadin P31-49 (19mer) (Zimmer, K. P., et al. 2010) are known to be held longer in the early endosomal vesicles as they delay early to late endosomal maturation and vesicular trafficking of several cargos, including EGFR which results in prolonged EGFR activation. Notably, CFTR malfunction also delays EGFR trafficking in bronchial epithelial cells (Villella, V. R., et al. 2013. Consistent with its capability to impair CFTR function (as shown in FIG. 1a), P31-43 reduced the total cellular abundance of BECN1 (FIG. 6a,b), hVps34 (FIG. 6c,d) and UVRAG (FIG. 6e), as it decreased small GTPases Rab5 and Rab7 levels in endosomal protein fractions (FIG. 60. These effects were counteracted by the CFTR potentiators VX-770 and VX-532 (FIG. 6a-f), unless the PI3K inhibitor 3-methyl-adenine (3-MA) (FIG. 6e) or the CFTR inhibitor CFTRᵢₙₕ₇₂ were added to the system. Consistent with the ability of TG2 to control endocytosis of surface receptors, TG2 was detected in the endosomal protein fraction from polarized Caco-2 cells pulsed with P31-43 as soon as after 15 min following internalization (FIG. 6g). In addition, immunoprecipitation of CFTR from early endosomal protein fractions revealed the presence of biotinylated P31-43 (FIG. 6h), suggesting that TG2 may allow P31-43 to encounter the NBD1 domain of CFTR in the early endosomes during CFTR recycling (Villella, V. R., et al. 2013). Notably, CFTR and P31-43 co-immunoprecipitated in clathrin⁺ EEA1⁻ plasma membrane protein fractions from Caco-2 cells as soon as after 5 min following incubation with P31-43, supporting the hypothesis that P31-43 may encounter and bind CFTR in clathrin⁺ PM fractions that also contain TG2 (FIG. 6i). Indeed, CFTR, TG2 and P31-43 all enter the endosomal compartment through clathrin⁺ vesicles for either recycling or lysosomal degradation (Lukacs et al, 1997; Zimmer, K. P, 2010).

Altogether, these results indicate that CFTR malfunction mediates the multifaceted effects of P31-43 on endosomal trafficking.

### Example 7

CFTR Potentiators Protect Epithelial Cells from CFTR Plasma Membrane Disposal Induced by P31-43.

Consistent with the capacity to decrease BECN1 and PI3K complex 3 activity, P31-43 increased the abundance of the plasma membrane (PM)-associated pool of the autophagic substrate SQSTM1/p62 (FIG. 7a) (Villella, V. R., et al. 2013; Luciani, A. et al. 2010; Kroemer, G. et al. 2010), an ubiquitin binding protein that favours the disposal of PM resident CFTR in bronchial epithelial cells upon functional CFTR inhibition (Villella, V. R., et al. 2013). Accordingly, P31-43 promoted the carboxy-terminal-of-hsp70-interacting-protein (CHIP)-mediated CFTR ubiquitilation (Villella, V. R., et al. 2013) (FIG. 7b), thus favouring SQSTM1/p62mediated reduction of the overall abundance of PM-associated CFTR after 24 h of challenge (FIG. 7c), an effect counteracted by both VX-770 and VX-532 (FIG. 7d).

### Example 8

CFTR Potentiators Protect Epithelial Cells from Innate Immune Activation Induced by P31-43.

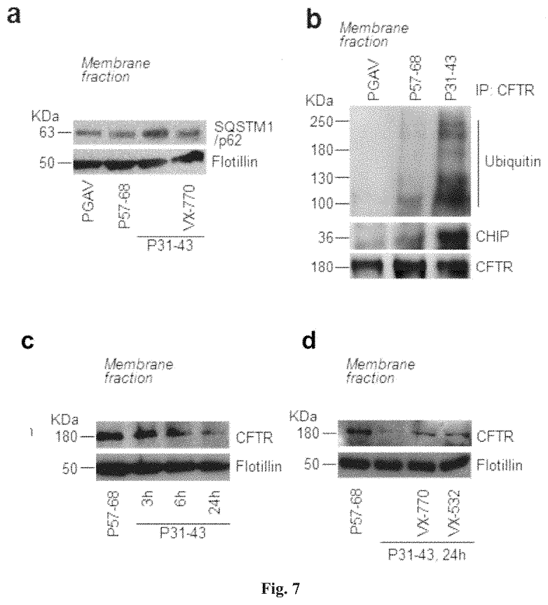

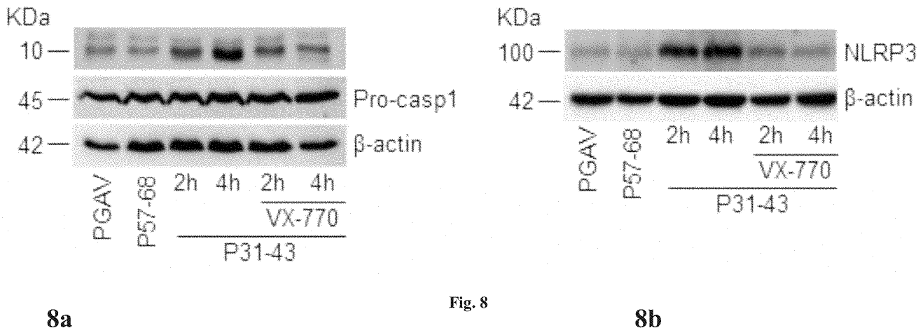

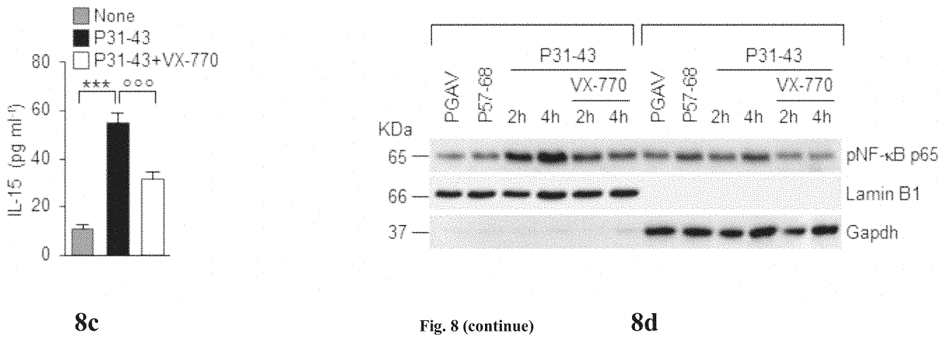

The activation of the NLRP3 inflammasome activity plays a major role in setting off inflammatory reactions (Palova-Jelinkova, L., et al. 2013). VX-770 prevented the capability of P31-43 to induce NLRP3 expression (FIG. 8a) and caspase-1 cleavage (FIG. 8b) in Caco-2 cells.

IL-15, a master pro-inflammatory cytokine produced by different cell types, including enterocytes and intestinal lamina propria cells, is a major trigger of CD and acts as a danger signal upon cellular distress (Jabri, B. et al. 2015). The Applicant demonstrates that VX-770 prevented the P31-43-induced production of IL-15 by Caco-2 cells (p<0.001) (FIG. 8c). Consistent with the presence of an active NF-κB binding motif in the IL-15 gene promoter, NF-κB p65 translocation into the nucleus was observed in Caco-2 cells after incubation with P31-43, and this effect was suppressed by VX-770(FIG. 8d).

### Example 9

CFTR Potentiators Protect In Vivo Gliadin-Sensitive Mice from the Effects of Gliadin.

Next, the Applicant administered gliadin to mouse models of gliadin sensitivity.

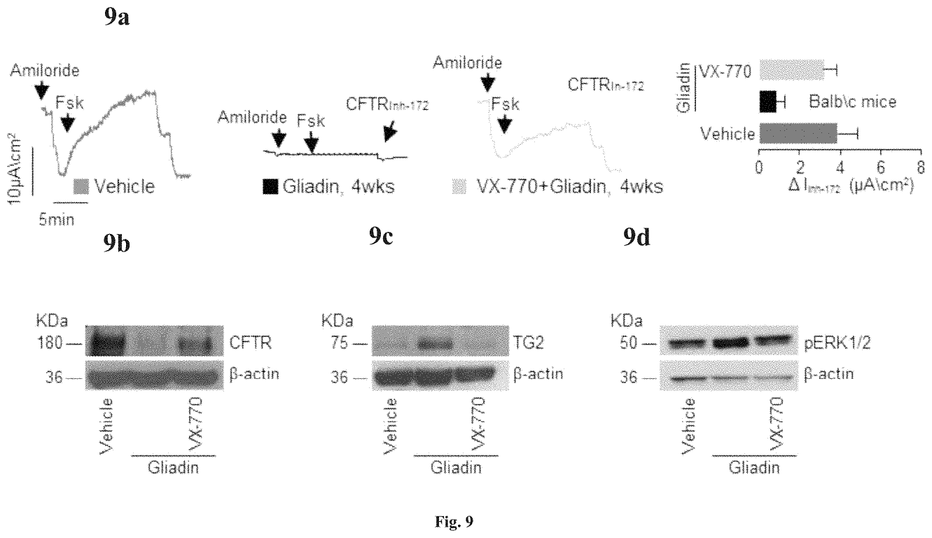

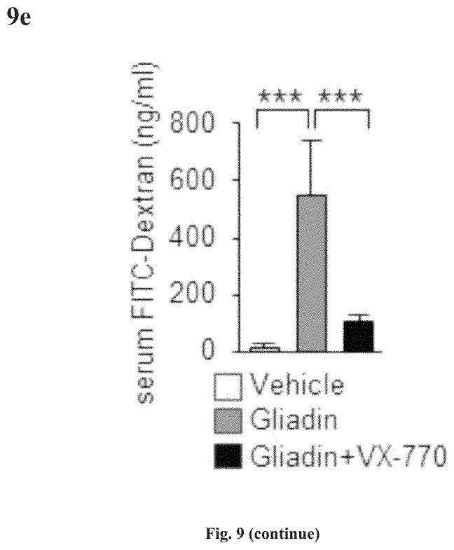

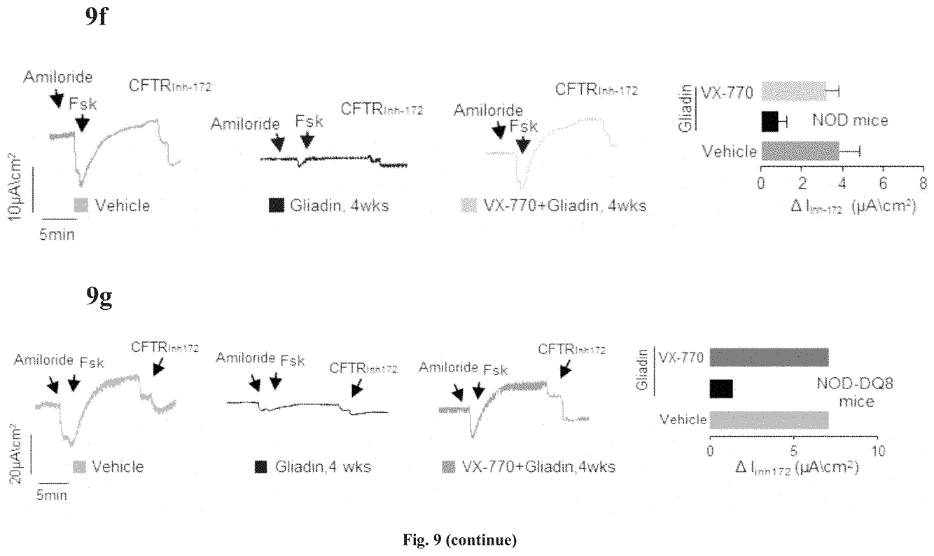

In the first model, BALB/c mice are fed for at least three generations with a gluten-free diet, followed by a 4-week gliadin challenge, as described (Papista, C., et al. 2012). Ten week-old BALB/c mice were orally challenged for 4 weeks with gliadin or vehicle in the presence or absence of 2 mM VX-770 administered intraperitoneally 15 min prior to gliadin challenge (n=10 per treatment group). In all tested mice, gliadin induced a VX-770-inhibitable decrease of CFTR function in small intestines (FIG. 9a) and reduced the abundance of CFTR protein (FIG. 9b). Moreover, VX-770 opposed the ability of gliadin to increase TG2 protein levels (FIG. 9c) and ERK 1/2 phosphorylation (FIG. 9d). VX-770 counteracted the ability of gliadin to increase intestinal permeability in vivo (FIG. 9e).

In the second model of gluten sensitivity, the Applicant resorted to non-obese diabetic (NOD) female mice, which spontaneously develop autoimmune type-1 diabetes (Maurano, F., et al. 2005). When orally administered to these mice, gliadin caused a VX-770-inhibitable reduction in CFTR function (FIG. 9f). Notably, the Applicant confirmed the CFTR inhibitory effects of gliadin in NOD mice transgenic for the CD-predisposing HLA molecule DQ8 (NOD-DQ8)(Galipeau, H. J, et al. 2011) (FIG. 9g). Altogether, the aforementioned results indicate that gliadin can inhibit intestinal CFTR function in vivo and that, in turn, CFTR malfunction predisposes to gliadin-induced inflammatory reactions.

### Example 10

CFTR Potentiators Prevent Gliadin-Induced Immune Dysregulation In Vivo.

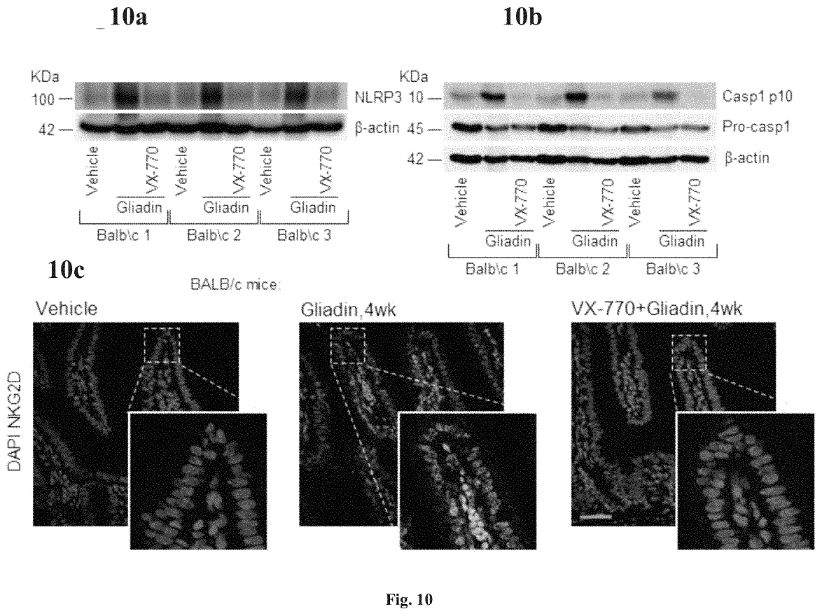

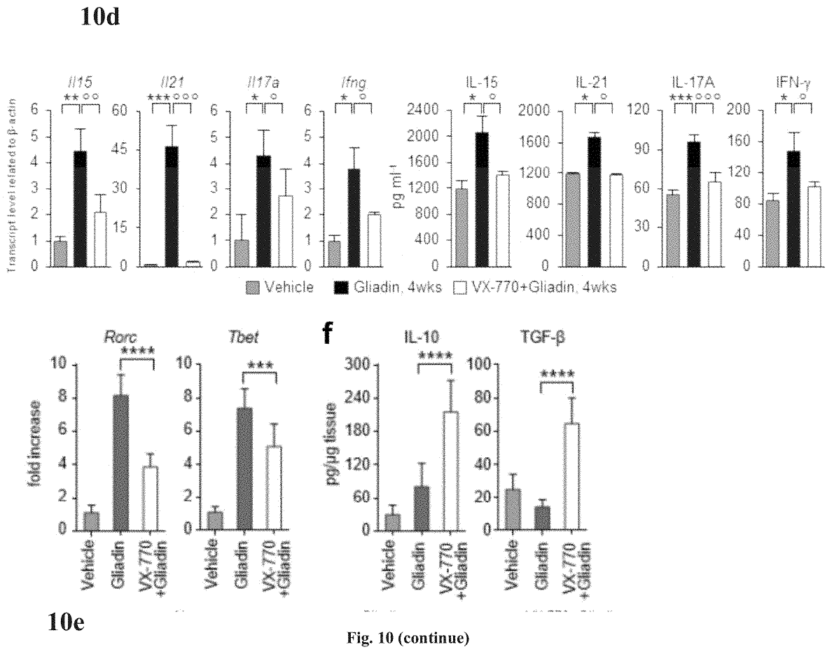

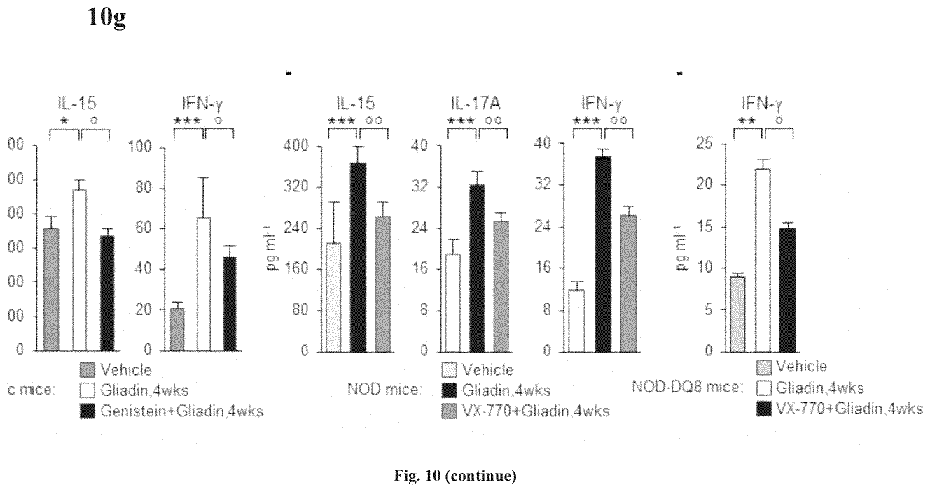

In the next step, the Applicant determined whether VX-770 would be efficient in protecting gliadin sensitive mice from the gliadin-induced immunopathology. VX-770 counteracted gliadin-induced NLRP3 expression (FIG. 10a) and caspase 1 cleavage (FIG. 10b), and the IL-15 dependent increased expression of natural killer (NK) receptor NKG2D by intraepithelial lymphocytes (FIG. 10c). VX-770 prevented the gliadin-induced increase of IL-15, IL-17A, and IFN-γ mRNA and protein (p<0.001) (FIG. 10d) and inhibited the gliadin induced expression of IL-21, which is known to positively correlate with Th1 and Th17 activity (FIG. 10d). Accordingly, both the Th17 (Rorc) and Th1 (Tbet) transcription factors were downregulated upon VX-770 treatment (FIG. 10e). In contrast, VX-770 restored the impaired IL-10 and TGF-β production in gliadin-sensitive mice (FIG. 10f). Similarly to VX-770, the CFTR potentiator genistein was effective in controlling cytokine upregulation in gliadin-sensitive BALB/c mice (FIG. 10g). The immunoprotective effects of VX-770 were confirmed in NOD mice challenged with gliadin after diabetes onset (FIG. 10h). Importantly, VX-770 counteracted the gliadin-induced increase of IFN-γ in NOD-DQ8 mice (FIG. 10i). In conclusion, it appears that the CFTR potentiator VX-770 can reduce epithelial stress and local immune dysregulation induced by gliadin.

### Example 11

CFTR Potentiators Oppose the Gliadin-Induced Immune Response Ex-Vivo in Celiac Patients.

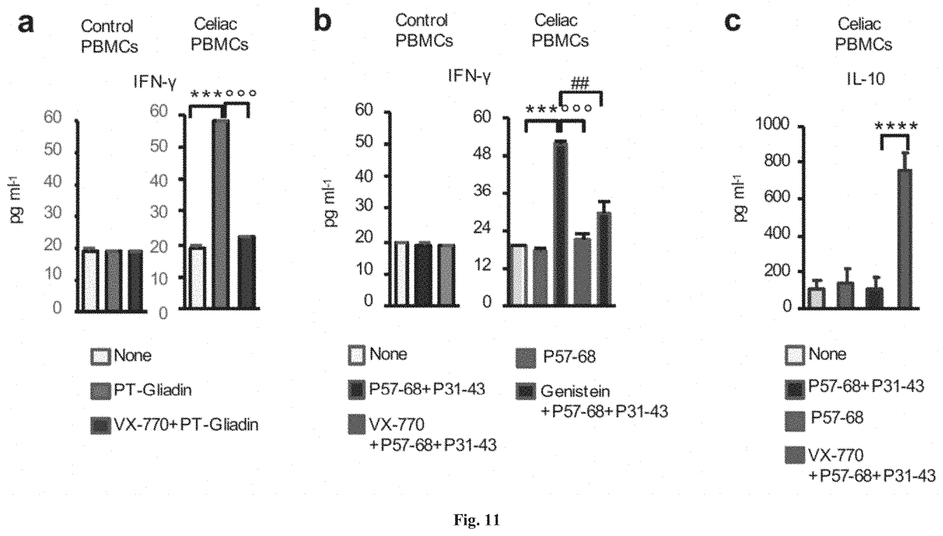

To translate our findings into the relevant clinical setting, we determined whether CFTR potentiators would prevent the HLA-restricted immune response to gliadin by peripheral blood mononuclear cells (PBMC) collected from celiac patients. PBMC collected from 6 celiac patients and 4 healthy controls, were cultured in the lower compartment of a bidimensional co-culture model in which confluent Caco-2 cells were placed in the upper compartment. Then, Caco-2 cells were challenged with PT gliadin or with a combination of P31-43 and P57-68 (or P57-68 alone as a negative control), as described, and IFN-γ and IL-10 were quantified in the supernatants from the lower compartment. In this system, both PT gliadin and the combination of P31-43 and P57-68 (but not the P57-68 alone) induced IFN-γ production in the lower compartments in which celiac, but not control, PBMC were present. VX-770 and genistein prevented the production of IFN-γ induced by the combination of peptides while they restored IL-10 levels (p<0.01) (FIG. 11 a-c). Notably, the challenge of Caco-2 cells with either PT-gliadin or the peptide combination in the bidimensional culture model led to IL-15 accumulation in the lower compartment (p<0.001 vs medium alone) regardless of the presence of PBMCs, an effect that was prevented by VX-770 (p<0.001 vs peptide combination).

Altogether, these preclinical results suggest that CFTR potentiators might be used for reducing pathogenic inflammatory reactions in patients with CD.

As detailed in the Examples, the findings reported above shed light on a yet unsolved issue of the celiac puzzle by showing how gliadin peptides overcome natural host defenses to trigger a stress/innate immunity activation in the small intestine. The Applicant has identified CFTR as the missing link between gluten and celiac intestine by showing how gliadin can impair CFTR protection to detune major mechanisms of mucosal defenses.

The Applicant has discovered that potentiators of CFTR channel gating represent a novel unforeseen option to treat celiac patients and demonstrates how CFTR potentiators are effective in counteracting the damaging effects of gliadin.

Gluten may often cause discomfort in a still undefined growing proportion of non-celiac individuals, irrespective of HLA susceptibility. The Applicant's finding demonstrates that Ivacaftor and other CFTR potentiators are a novel unforeseen attractive option also for the treatment of individuals affected by non-celiac gluten-sensitivity (NCGS).

Methods

Peptides

The following peptides were synthesized by Inbios (Napoli, Italy): α-gliadin peptide LGQQQPFPPQQPY (P31-43-SEQ ID 1) or QLQPFPQPQLPY (P57-69-SEQ ID 2) or scrambled GAVAAVGVVAGA (PGAV-SEQ ID 3) or modified P31-43 (different Q-A or P-A substitutions, single position or double positions, 4QA, SEQ ID 4). All peptides were obtained with or without Biotin-NH2-tag.

Molecular Modelling

NBD1 and NBD2 crystal structures were retrieved from Protein Data Bank (PDB: 2BBO and 3GD7). All the ligands and cofactors were removed; hydrogen atoms were added to the protein structure using Autodock 4.2. To minimize contacts between hydrogens, the structures were subjected to Amber force field keeping all the heavy atoms fixed. Peptides P31-43 (LGQQQPFPPQQPY) 4QA (LGQAAPFPPAAPY), were built using PEP-FOLD3, by generating 5 clusters sorted using sOPEP energy value.

Protein-Protein docking analysis was performed using two FFT-based docking software PIPER and Zdock (Chen, R., Li, L. et al. 2003). The procedures were performed using NBD1 or NBD2 crystal structures as the target proteins while the peptides were considered as probes. 1000 complexes were obtained from both docking algorithms and clusterized using the pairwise RMSD (Root Mean Square Deviation) into 6 clusters. The final complex was chosen according to the energy scoring function. In particular, among the 5 clusters generated by PEP-FOLD3 one single P31-43 peptide conformation resulted to be selected efficiently by both Protein-Protein docking algorithms (75% of total conformations). Unfortunately, the Protein-Protein docking analysis was not able to retrieve significant P31-43 peptide conformations able to interact with NBD2; indeed the best solution selected presented a very low sample percentage (7%). P31-43 conformation bound to NBD1 was optimized by a remodeling of the peptide folding in close contact with NBD1 (PEP-FOLD3), starting from the patch of interaction retrieved from the Protein-Protein docking procedure.

Molecular dynamics (MD) simulations of the final complex (parameterized with AMBER14SB/ff14SB were performed with ACEMD (Accellera MC4-node, 4× GeForce GTX980 GPUs) in order to verify the complex stability over time; in particular a 500 ns of NPT (isothermal isobaric ensemble, 1 atm, 300K) MD simulation was performed after an equilibration phase of 10 ns (positional restraints were applied on carbon atoms to equilibrate the solvent around the protein).

The interaction pattern between P31-43 and NBD1 obtained from the computational approach described above, was used to design a peptide unable to efficiently interact with NBD1. In particular an in silico Ala-scanning was performed targeting the P31-43 residues mostly involved in the interaction with NBD1. All the peptides produced by the combination of different amino acid substitutions were subjected to PEP-FOLD3 protocol and docked against NBD1 through the combined PIPER/Zdock approach. The peptide presenting an Ala substitution in 4th, 5 h, 10th and 11th positions (4QA) was selected as the best solution as control peptide unable to interact with NBD1 (sampling percentage of only 12% compare to the 75% of P31-43).

The same Protein-Protein docking and Molecular Dynamics approaches were used to investigate the binding of P31-43 to CFTR structure obtained via Electrom Microscopy (PDB code: SUAK), using the parameters as described before. In this particular case and during Molecular Dynamics processes, the transmembrane domains and the R-Domain were kept fixed, the first being immersed in a membrane system, the latter because its position is not completely determined in the structure.

In Vitro Studies

Proteins Cloning, Expression and Purification

Purified human NBD1 was generously provided by Dr.

C. G. Brouillette (University of Alabama at Birmingham, Ala., USA). Plasmid encoding human recombinant NBD1 domain was obtained by DNASU plasmid repository (clone ID: HsCD00287336) and the two double mutants, namely F400A/E403A and P439A/P477A, were purchased by Primm (Milan, Italy). All the clones contained six histidines in the N-terminus to facilitate the purification following the protocol reported in www.cftrfoldingconsortium.com.

Surface Plasmon Resonance

Peptide-protein interactions were studied using a Biacore™ T100 (GE Healthcare) instrument. hrNBD1 domain or the indicated biotinylated peptides were immobilized by thiol coupling on a CMS (series S) sensor chip to a final density of 2000 resonance units (RU) or by biotin-streptavidin capture on a SA (series S) sensor chip to a final density of 250 RU, respectively. A flow cell with no immobilized protein or peptide was used as control. Binding analysis was carried out at 20° C. in a running buffer consisting of 10 mM HEPES, pH 7.4, 150 mM NaCl, 0.05% (v/v) Tween-20, 2 mM ATP, 5 mM MgCl2, 1 mM DTT applying a flow rate of 20 μl/min. For each experiment a Biacore™ method program was used. It included a series of three start up injections (running buffer), zero control (running buffer) and different concentrations of ligand. In all cases, the chip surface was regenerated with two 20 s injections of 0.03% (w/v) SDS; this treatment restored the baseline to the initial RU value. Each sensorgram (time-course of the surface plasmon resonance signal) was corrected for the response obtained in the control flow cell and normalized to baseline. Data were analysed using the 2.0.3 BIAevaluation software (GE Healthcare). At least three independent experiments for each analysis were performed.

Peptide-protein interactions were further studied exploiting a different biochemical strategy: recombinant NBD1, both wild type and/or its mutants, were incubated at 4° C. for 30′ in 40 mM Tris pH 7.5 in the presence of the biotinylated P31-43 peptide unless otherwise indicated and resolved in native conditions by BN-PAGE (Novex, Invitrogen). The complexes were then blotted onto PVDF membrane in accordance with the manufacturer's instructions and the interacting partners were detected with streptavidin-HRP antibody (PerkinElmer).

In Vitro Characterization of NBD1 ATPase Activity

To assess the capability of P31-43 peptide to bind NBD1 domain and modify its ATP binding site the intrinsic Tryptophan fluorescence of NBD1 was measured. Experiments were performed in a 0.2 ml fluorimeter cuvette at 25° C. using Varian Cary Eclipse fluorescence spectrophotometer. NBD1 protein was diluted to 1 μM in a buffer containing 50 mM Tris/HCl (pH 7.6), 150 mM NaCl, and 5 mM MgCl2, supplemented with 2 mM ATP and P31-43 5 μM when indicated. All spectra were corrected for buffer fluorescence. Excitation wavelength was adjusted to 292 nm and emission was scanned over the range 300-500 nm.

The measurement of NBD1 ATPase activity was performed with a malachite green based kit supplied by Sigma (cod. MAK113) in accordance with the manufacturer's instructions. The enzyme activity was measured at 620 nm at room temperature using a Multiskan 96 well plates reader.

Cells and Treatments

Human colon adenocarcinoma-derived Caco-2 and T84 cells were obtained from the ATCC. Cells were maintained in T25 flask in Modified Eagle Medium (MEM) for Caco-2, or Ham's F12+DMEM (1:1) for T84, supplemented with 10% fetal bovine serum (FBS), 2 mM Glutamine+1% Non Essential Amino Acids (NEAA) and the antibiotics penicillin\streptomycin (100 units/ml) (all reagents from Lonza) (Luciani, A., et al. 2010. Cells were grown in Transwells (Corning, 3470 or 3460) under the normal condition (Luciani, A., et al. 2012). Briefly, 8×10⁴ or 5×10⁵ cells were seeded in 6.5-mm diameter or 12-mm diameter, respectively, and grown until the RT reached 800 to 1,200 Ω·cm2. Transwells with a pore size of 0.4 μm were used. Medium in both the apical and basolateral chambers was changed every other day. Cells were treated with 20 μg/ml of either α-gliadin peptide P31-43 or P57-69 or scrambled PGAV or modified P31-43 either biotin-tagged or not, for different time point (from 1 h short challenge up to 24 h). Cells were also treated with 50 or 100 μg/ml of P57-68 or PGAV peptides for 3 h. Caco-2 or T84 cells were also treated with: CFTR potentiators VX-770 (10 μM) or VX-532 (20 μM) (Selleck chemicals) or Genistein (10 μM) (Sigma-Aldrich) or in presence or absence of CFTR inhibitor172 (CFTRᵢₙₕ₁₇₂, 20 μM) or 3-methyl-adenine (3-MA, 20 μM) (Calbiochem), Z-DON (20 nM, Zedira) or BAPTA-AM (10 μM, Calbiochem).

Mice and Treatments

BALB/c mice (background BALB/cAnNCrl) were purchased from Charles River (Varese, Italy). Three generation gluten-free diet (Mucedola srl, Milan), male and female, were challenged with gliadin for 4 weeks. To assess the effects of VX-770 into a controlled environment, mice were challenged via gavage for 4 weeks with i) vehicle alone or ii) gliadin (SigmaAldich, G3375) (5 mg/daily for one week and then 5 mg/daily thrice a week for 3 weeks)(Moon, S. H., et al. 2016) in the presence or absence of intraperitoneal VX770 (0.075 mg/mice in 100 μl DMSO, 2 mM, SelleckchemS1144) or genistein (25 mg\kg in 100μ1 DMSO, SigmaAldrich), administered 15 minutes prior gliadin challenge (n=10 mice per group of treatment).

CF mice homozygous for the F508del-CFTR in the FVB/129 outbred background (Cftrtm1EUR, F508del, FVB/129, abbreviated Cftr^{F508del/F508del}) and Wild Type littermates, male and female, were obtained from Bob Scholte, Erasmus Medical Center Rotterdam, The Netherlands, CF coordinated action program EU FP6 LSHMCT-2005-018932. Mice were treated as in i) or ii).

Transgenic KO Cftr mice (B6.129P2-KOCftrtm1UNC, abbreviated Cftr^{-/-}), and Wild Type littermates, were purchased from The Jackson Laboratory (Bar Harbor, Me., USA). The heterozygous Cftr^{F508del/+} males were backcrossed with the heterozygous Cftr^{+/-} females to obtain F508del/null CFTR heterozygous mice (abbreviated CftrF508del/−).

In order to obtain TG2−/− mice carrying F508delCFTR mutation, C57Bl/6 mice KO for TG2 (obtained from Gerry Melino, Department of Experimental Medicine and Biochemical Sciences, University of Rome ‘Tor Vergata’, Rome, Italy) were crossed with 129/FVB mice heterozygous for F508del mutation (abbreviated Cftr^{F508del/+}/TG2^{-/-}). The newly generated mice were housed at Department of Experimental Medicine and Biochemical Sciences, University of Rome ‘Tor Vergata’(Rome, Italy).

All above described mice for the study were aged 10-week-old. At least ten mice per group per experiment were used.

Prediabetic NOD (Non-obese diabetic) mice were purchased from Charles River (Varese, Italy). Diabetes incidence was followed weekly measuring of blood glucose levels with a Contour glucose meter (Bayer; US). At time 12-13 weeks, female mice with manifested diabetes incidence (>250 mg\dl), were challenged as described in i) or ii).

NOD.scid AB0nullDQ8 mice (NOD DQ8tg, transgenic mice that express HLA-DQ8 in an endogenous MHC class II-deficient background were backcrossed to NOD mice for 10 generations and intercrossed to produce congenic NOD AB DQ8 mice) were purchased from The Jackson Laboratory (Bar Harbor, Me., USA) and were weaned and maintained on a low-fat (4.4%), gluten-free diet (Mucedola srl, Milan) and bred in a conventional, specific pathogen-free colony at the San Raffaele Scientific Institute SOPF animal house (Milan, Italy). Mice were challenged as described in i) or ii).

At the end of the last daily treatment, mice were anesthetized with Avertine (tribromoethanol, 250 mg/kg, Sigma Aldrich, T48402) and then killed; the intestines were collected for CFTR function analysis or stored for all described techniques.

These studies and procedures were approved by the local Ethics Committee for Animal Welfare (IACUC No 583, 713, 661, 628) and conformed to the European Community regulations for animal use in research (2010/63 UE).

Purification of PBMC and Transwell Co-Culture Model

Five ml of peripheral blood have been withdrawn from 8 untreated celiac patients (females and males, age range 8-25 years) and from 3 DQ2+ positive not CD affected controls (healthy first-grade relatives of celiac patients). The Ethics Committee of ISS approved the protocol (# CE/12/341), and patients or patients' parents signed the informed consent. Peripheral blood mononuclear cells were isolated using lympholite (Cederlane, UK) density gradient overlaid by heparin blood diluted 1:1 in PBS and centrifuged (20 min at 900 rpm). After being washed three times, PBMCs were resuspended in complete RPMI 1640 supplemented with 25 mM HEPES, 10% (v/v) heat-inactivated FBS, 100 U/ml penicillin, 100 mg/ml streptomycin, and 1% 2 mM 1-glutamine.

For transwell experiments using polarized Caco-2 cells, 3 weeks prior to the experiment, Caco-2 cells were seeded at a density of 80×10³ cells×cm² on 0.4-μm, 1-cm² tissue culture inserts (Costar, Corning Incorporated). Transwell cultures (12-well) with confluent Caco-2 monolayers were used for co-culture with 1 ml PBMC (1.5×10⁶ cells/ml) using PBMC medium and kept in an incubator at 37° C. and 5% CO₂. Cells were allowed to settle for 1 h before the starting of the experiment. The permeability of the epithelial monolayer was assessed just before the experiments, measuring the transwell electrical resistance between the upper and lower compartments. A value of transwell resistance >800 Ω×cm² has been considered index of a fully formed epithelial monolayer, not allowing the paracellular passage of molecules⁶⁴.

Caco-2 cells were apically exposed for 3 h with P31-43 peptide (20 μg/ml) and then treated with P57-68 (20 μg/ml) in presence or absence of CFTR potentiators VX-770 or Genistein. As negative control, cells were treated with medium alone, and with P57-68 alone. After the treatments, supernatants from the basolateral compartment were collected, centrifuged, and stored at −20° C. until cytokine measurement. At the same time, the cells from the apical compartment were harvested, lysated, and stored at −80° C.

Using Chamber

Chambers for mounting either transwell cell cultures or mouse tissue biopsies were obtained from Physiologic Instruments (model P2300, San Diego, Calif., USA). Chamber solution was buffered by bubbling with identical Ringer solution on both sides and were maintained at 37° C., vigorously stirred, and gassed with 95% O₂/5%. Cells or tissues were short circuited using Ag/AgCl agar electrodes. A basolateral-to-apical chloride gradient was established by replacing NaCl with Na-gluconate in the apical (luminal) compartment to create a driving force for CFTR-dependent Cl secretion. To measure stimulated Isc, the changed sodium gluconate solution was supplied with 100 μM amiloride. Agonists (forskolin) were added to the bathing solutions as indicated (for a minimum 5 min of observation under each condition) to activate CFTR channels present at the apical surface of the epithelium (either cell surface or lumen side of the tissue) and CFTR_{Inh-172} (10 μM) was added to the mucosal bathing solution to block CFTR-dependent Isc. Shortcircuit current (expressed as Isc (μA/cm2)) and resistance were acquired or calculated using the VCC600 transepithelial clamp from Physiologic Instruments and the Acquire & Analyze 2.3 software for data acquisition (Physiologic Instruments), as previously described Tosco A, et al. 2016; Romani, L. et al. 2017).

Permeability Assay: Fluorescein Isothiocyanate-Dextran 4000 (FITCD4000) Test

The FITC-D4000 test in treated BALB/c mice (as described in mice and treatments section) was performed as previously described (Volynets, V., et al. 2016)

Methods are detailed in Supplementary Information.

CRISP/CAS9 CFTR Knockout

CFTR CRISP/CAS9 KO plasmids were purchased from Santa Cruz Biotechnology and transfected in Caco-2 cells by UltraCruz transfection reagent according to the manufacturer's instructions (Santa Cruz Biotech.). Successful transfection of CRISPR/Cas9 KO Plasmid was visually confirmed by detection of the green fluorescent protein (GFP) by immunofluorescence. The cells were then sorted by replacing selective media with Puromycin antibiotic approximately every 2-3 days for a minimum of 3-5 days. The knockout was then confirmed by western blot with specific CFTR antibody and by functional assay (Ussing chamber or SPQ assay).

Immunoblot and Immunoprecipitation

The whole lysate or membrane fraction proteins of cell lines and mice intestine homogenates were obtained from treated and untreated cells or mice as described. The equal amount of protein were resolved by SDS-PAGE gel and blotted with antibodies against: SQSTM1, (Sigma Aldrich, 108k4767) 1:1000, PPARγ (Santa Cruz Biotechnology, sc7273) 1:500, BECN1 (Abcam, ab58878) 1:1000, CFTR clone M3A7 (Abcam, ab4067) 1:500, phospho-ERK1/2 (php42/44, Cell Signaling Technology, #91101) 1:1000, UVRAG (Santa Cruz Biotechnology sc8215) 1:1000, NHERF-1 (BD, 611161) 1:1000, EZRIN (BD, 610603) 1:1000, hVps34 (SigmaV9764) 1:300, biotin (Abcam, ab1227) 1:2000, PKAr2a (BD, 610625) 1:500, Ubiquitin (Cell Signaling clone P4D1, #3936) 1:1000, CHIP (Calbiochem PC711) 1:500, Caspase-1 p10 (M-20) (Santa Cruz Biotechnology, sc-514) 1:200, NLRP3 (Abcam, ab4207), 1:500, IDO1 (monoclonal; cv152) 1:1000^{[80]}, used as primary antibodies. Normalization was performed by probing the membrane with anti-β-actin (Cell Signaling, #4970) 1:1000, anti-Gapdh (Sigma-Aldrich, G8795) 1:1000 and anti-flottilin (Abcam ab15148) 1:1000 antibodies. Immunoprecipitations of endogenous proteins were performed on 500 μg of lysates from Caco-2 cells. The proteins were incubated for 12-18 h at 4° C. with 10 ug\ml of specific antibody followed by the addition of Protein A/G-agarose beads for 4 h at 4° C. The beads were then washed three times with lysis buffer and Laemmli's sample buffer was added to the samples. The membranes were then blotted with: CFTR clone CF3 (Abcam, ab2784) 1:1000, TG2 (clone CUB7402; NeoMarkers) 1:1000, CHIP (Calbiochem PC711) 1:500, Ubiquitin (Cell Signaling clone P4D1 3936), isopeptide (Abcam 422) 1:1000, PKAR2 (BD 610625) 1:500.

For detection of complex with biotnylated-P31-43 or immune-complex of glutamime-lysine bonds, the beads were resolved in non-reducing and non-denaturating conditions, and then blotted with Streptavidin-HRP (Sigma 52438) 1:3000 or anti-biotin (Abcam ab2103) 1:2500 and anti-isopeptide (Abcam 422) 1:1000.

Nuclear and Cytoplasmic Extraction

For cytoplasmic and nuclear extracts human colorectal carcinoma Caco-2 cells were harvested, washed in cold PBS twice, and centrifuged at 6000 rpm for 5 min in cold room to collect pellets. Pellets were resuspended in double cell volume of cytoplasmic extract (CE) buffer (10 mM HEPES, pH 7.9, 10 mM KCl, 0.1 mM EDTA, 0.3% NP-40 and 1× protease inhibitor cocktail (Thermo Scientific™)), incubated on ice for 10 min and centrifuged at 3000 rpm for 5 min to obtain the supernatants as cytoplasmic fraction. The pellets (containing the nuclei) were resuspended in equal volume of nuclear extract (NE) buffer (20 mM HEPES, pH 7.9, 0.4 M NaCl, 1 mM EDTA, 25% Glycerol and 1× protease inhibitor cocktail), incubated on ice for 10 min and centrifuged at 14,000 rpm for 5 min to obtain the supernatants as nuclear fraction. The protein concentrations were measured by Bradford protein assay (Protein Reagent, Bio-Rad). For Western blots, cytoplasmic and nuclear proteins were fractionated on 10% SDS-PAGE and transferred to Nitrocellulose blotting membranes (AmershamTMProtran™ 0.45 μm NC, GE Healthcare, Life science). Membranes were incubated with antiphospho-NF-κB p65 (Ser536) ((93H1), Cell Signaling, #3033), 1:1000, Lamin Bl-Nuclear Envelope Marker (Abcam, ab16048) 1:5000, anti-β-actin (Cell Signaling, #4970) 1:1000.

Immunofluorescence Assay

Tissue sections and cells were processed as previously described. Mouse tissues: briefly, tissue sections were fixed and then incubated with the following primary antibodies: CD71 (Abnova, abv25484) 1:300 or CD71 (Thermo-Fischer, #136800) 1:300, IgA-FITC (mouse Sigma-Aldrich) 1:20, TG2 (clone CUB7402; NeoMarkers) 1:300, FOXP3 (Abcam, ab54501), 1:500, Occludin (Invitrogen, 71-1500) 1:150, IL10 (Abcam, ab9969) 1:200. Cell lines: the cells were fixed after challenge with P31-43 i) for 3 h at 37° C. and then incubated with Lampl (Abcam, 24270) 1:300 and Alexa-546 Streptavidin 1:300 or ii) for 15 minutes at 37° C. and then incubated with UVRAG (Santa Cruz, sc8215) 1:100 and EEA-1 (Abcam, 2900) 1:300 or TG2 (clone CUB7402) 1:500 and Phalloidin-Alexa488 conjugated (Thermo-Fischer, A12379) 1:500 or EEA-1. All primary antibody were incubated over-night at 4° C. After washing, the slides were incubated with Alexa-Fluor-488 or 546 secondary antibodies (Molecular Probe) 1:300. Moreover, IgA staining in Caco-2 cells was performed as described: P57-68 or P31-43 peptides were added for 2 h in the apical compartment of polarized Caco-2 cell after 1 h at 4° C. of pre-incubation with colostrum derived S-IgA (250 μg\ml, Sigma-Aldrich). Cells were washed, fixed and stained with anti-human IgA-FITC (Sigma-Aldrich, F5259) 1:50 and CD71. Slides were washed, mounted on glass coverslips using ProLong Gold (Invitrogen), and imaged on a Zeiss LSM 510 confocal laser-scanning microscope (Carl Zeiss MicroImaging) equipped with x63 oil immersion or x40 or x20 objective. All of the experiments were repeated at least three times, and representative images are shown. To quantify the levels of co-localization, confocal serial sections were acquired from 8-10 cells per experimental condition, exported in TIFF format and processed as previously described¹⁶. The immuno fluorescence of GFP-FYVE_{SARA} spots were performed as described (Villella, V. et al. 2013) and fully described in Supplementary Information.

Statistical Analysis

GraphPad Prism software 6.01 (GraphPad Software) was used for analysis. Data are expressed as means±SD. Horizontal bars indicate the means. Statistical significance was calculated by ANOVA (Bonferroni's post hoc test) for multiple comparisons and by Student's t test for single comparisons. The Applicant considered all P values 0.05 to be significant. The in vivo groups consisted of ten mice/group. The data reported are either representative of at least three experiments.

Transient Transfection and RNA Interference

Cells were transfected with TG2 siRNA or CFTR siRNA or scrambled oligonucleotides by using Lipo RnaiMax (ThermoFischer) as described⁶⁻⁸. Cell lines were also transfected with pCMV-Tag2bFLAG-SQSTM1-ΔUBA (E396X) (kind gift of Dr. Lynne J. Hocking, University of Aberdeen, UK), and GFP-tagged FYVE_{SARA} domain (PtdIns3P probe) (kindly provided by S. Corvera) expression vectors as described (Villella, V. et al. 2013). Empty vectors were used as control.

GFP-Tagged FYVE_{SARA} Detection

Caco-2 cells were transfected with GFP-tagged FYVE_{SARA} domain plasmids and after 24 hours the cells were challenged with P31-43 in presence or absence of VX-770. After fixation with 4% PFA and saponin 0.2% permeabilization the cells were incubated with EEA1 (Abcam.ab2900) 1:300 overnight at 4° C.; after washing the Alexa-Fluor546 secondary antibody was added. Zeiss LSM 510 confocal laser-scanning microscope (Carl Zeiss MicroImaging) equipped with x63 oil-immersion or x40 objective were used and quantification of number of GFP-FYVESARA spots per cell was performed using the AnalySIS software (Soft Imaging Systems GmbH, Muenster, Germany).

Membrane Fractionation

Protein from membrane fractionation were obtained as described. Cells were homogenized with a PotterElvehjem pestle and centrifuged at 2300×g for 15 min at 4° C. Supernatants that contains the cytoplasmic and PM fractions were centrifuged 1 h at 16 000×g at 4° C.; the pellet was the intact membrane and was solubilized in BUFFER A (20 mM Tris-HCl pH 7.4, 2 mM EDTA, 20 mM 2-ME, 1×PMSF, 1 μg/ml inhibitor protease cocktail)+1% Triton X-100 and centrifuged 1 h at 60 000×g in the ultracentrifuge. The supernatants were collected as PM fraction. Equivalent amounts of proteins (500 mg) were used for Immunoprecipitation assay. Proteins of PM fraction were used for IP or WB and immunoblotted against CFTR, Ezrin, NHERF-1, SQSTM1 \p62, CHIP, Ubiquitin and Flottilin antibodies.

Differential Fractionation and Separation of Endosomes

The separation of endosomal fractions was performed as described⁹ from treated and untreated cells. Protein of endosomal fraction were resolved by SDS-Page and blotted with primary antibodies against Rab-5(Abcam 18211) 1:500, Rab-7(Abcam 77993) 1:500, EEA-1 (Abcam 2900) 1:500, TG2 or CFTR. EEA-1 was used as early endosome marker. For detection of complex with biotnylated-P31-43, the beads were resolved in non-reducing and non-denaturating conditions, and then blotted with Streptavidin-HRP (Sigma S2438) 1:3000 or anti-biotin (Abcam ab2103) 1:2500.

Limited Proteolysis of CFTR.

Membrane fraction (1-1.5 mg\ml protein) were digested in PBS buffer at the indicated concentration of trypsin for 15 min on ice as described. Proteolysis was terminated by 0.4 mg\ml soybean trypsin inhibitor (Sigma), 2 mM MgCl₂, 1 mM PMSF, 5 μg\ml leupeptin and pepstain. Digested membrane proteins were either dissolved in Laemmli's sample buffer for immunoblot analysis. The protease susceptibility of the full-length CFTR was measured by immunoblotting, densitometry and expressed as the percentage of remaining CFTR relative to the non-digested sample.

Halide Efflux Analysis

The analysis of halide efflux was performed by the iodide-sensitive fluorescent indicator, SPQ (Molecular Probes/Invitrogen), as previously described. The peak of halide efflux rate (usually after Fsk plus IBMX adding) of cells was calculated in accordance with the Stern-Volmer relationship. The rates were calculated using SigmaPlot Version 7.1 for each mean fluorescence trace for each time point generated from the 50 cells examined per population per coverslip.

TGM2 Enzyme Activity Detection

TG2 enzymatic activity in Caco-2, treated as described above, was detected: 1) by incubating unfixed sections with biotin-mono-dansylcadaverine (MDC, 10 mM, Molecular Probe) for 1 h at room temperature and then stained with Alexa-fluor488-Streptavidin, as previously described (JI2008/2009) or 2) or by incorporation of 5(biotinamido)pentylamines (BAP) into protein substrates. For BAP-incorporation, 2 mM BAP (Soltec Ventures, B110) were directly added into the medium together with the indicated treatments. In the presence of TG2 transamidating activity, BAP is incorporated into the substrates. To measure this activity, cells were lysed and proteins were resolved by SDS-polyacrylamide gel. The blots were incubated with antiBiotin antibody.

Permeability Assay: Fluorescein Isothiocyanate-Dextran 4000 (FITCD4000) Test

The FITC-D4000 test in treated BALB/c mice (as described in mice and treatments section) was performed as previously described. The 4000-Da test substance can be easily detected in blood plasma by a photometric approach. Briefly, FITC-D4000 (Sigma-Aldrich) was administered to the mice by gavage at a concentration of 600 mg/kg body weight and a volume of 200-300 μl, using a stock solution at 50 mg/ml. After gavage, the mice remained in the metabolic cages until the experiments were completed and the mice were killed. One hour after gavage, the animals were anesthetized and blood was taken by cardiocentesis, heparinized, and then centrifuged (10 min, 12,000 g, 4 C). Plasma was light protected and stored at −80° C. for photometric analysis of FITC-D4000. For use as a measurement, the plasma was diluted in an equal volume of phosphate buffered saline (PBS, pH 7.4). Standards (range 50-0.312 μg/ml) were obtained by diluting the FITC-D4000 gavage stock solution in PBS. An amount of 100 μl of both diluted animal samples and standards, as well as blanks (PBS and diluted plasma from untreated animals), were transferred to black 96-well microplates. Analysis for the FITC-D4000 concentration was carried out with a fluorescence spectrophotometer (Multi-Detection Microplate Reader) at an excitation wavelength of 485 nm and an emission wavelength of 528 nm.

Real-Time and Reverse Transcription Analysis

The analysis was performed as previously described.

Total RNA was extracted with the RNeasy Mini Kit (Qiagen, 74104) from mouse intestine homogenates. The mRNA was reverse transcribed with Onetranscript plus cDNA synthesis kit (abm good). Expression levels of genes were normalized to β-actin (primer design HK-sy-mo600) levels in the same samples.

ELISA

ELISA analysis was performed on tissue samples using standard ELISA kits (R&D Systems) for IL-15, IL-17A, INFγ IL21, IL10, TGF-β. According to the manufacturer's instructions. Samples were read in triplicate at 450 nm in a Microplate Reader (BioRad, Milan, Italy) using Microplate Manager 5.2.1 software. Values were normalized to protein concentration evaluated by Bradford analysis.

Reticulocyte Lysate System

To confirm the interaction of P31-43 to CFTR protein in a heterologous expression system, the Applicant expressed in vitro full length CFTR in a reticulocyte expression system according to manufacturer's instructions. Briefly, 1 μg of pcDNA-CFTR wild-type plasmide was incubated with mixed reagents of TnT® Coupled Reticulocyte Lysate Systems kit (Promega, L4611). To ensure the glycosylation of full length CFTR, the system was added with Canine Microsomal Membranes

The reaction was performed as described:
TnT® Reticulocytes Lysate 25 μl
TnT® Reaction Buffer 2 μl
TnT® RNA Polymerase (T7) 1 μl
Amino Acid Mixture, Minus Leucine, 1 mM 0.5 μl
Amino Acid Mixture, Minus Methionine, 1 mM 0.5 μl
RNasin® Ribonuclease Inhibitor, 40 u/μl 1 μl
pcDNA CFTR WT or Control DNA, (0.5 μg/μ1) 2 μl
Canine Microsomal Membranes 3 μl
Nuclease-Free Water to a final volume of 50 μl

Then, the reaction was incubated at 30-33° C. for 90 min to favour the translation processing of protein. At the end of the reaction, the samples were incubated with biotinylated P31-43 (20 μg\ml) in the presence or absence of VX-770 (10 μM) for 2 hours. The total lysates were resolved onto 8% polyacrilammide gel, after immunoprecipitation with CFTR antibody. The gel was transferred on PVDF filter and blotted with HRPStreptavidin or CFTR antibody. Empty control pcDNA plasmide was used as control.

### DETAILED DESCRIPTION OF THE FIGURES DISCUSSED IN THE EXAMPLES

FIG. 1: a. Representative traces of CFTR-dependent Cl-secretion measured by forskolin (Fsk)-induced increase of chloride current (Isc (μA/cm2)) in Caco2 cells mounted in Ussing chambers after 3 h of incubation with the α-gliadin-derived peptide P57-68 or P31-43 in the presence or absence of VX-770; quantification of the peak CFTR Inhibitor 172 (CFTRinh172)-sensitive Isc (ΔIsc) in Caco-2 cells (n=3-5). b-c. CFTR-dependent Cl-secretion measured as in a. (b) Caco-2, (c) T84 cells. d. Assessment of iodide efflux by SPQ fluorescent probe upon stimulation with forskolin (Fsk) expressed as percentage of CFTR function in the presence or absence of peptide challenge with or without treatment with CFTR potentiators with CFTR potentiators VX-770, VX-532 or genistein.

FIG. 2: a, Protein-Protein docking and Molecular Dynamics of P31-43 (violet) bound to NBD1 (orange). Left side: general view of P31-43 and NBD1 interaction. Upper right: detailed interaction pattern, highlighting the most important amino acids. Lower right: in silico NBD1/P31-43 complex compared to the original crystallographic positions of Tip 401 and ATP. b, Graphical view of the in silico sampling percentage of P31-43 against NBD1/NBD2 (left 10 panel), and of P31-43/4QA (4QA) against NBD1 (right panel). c-d, Surface plasmon resonance (SPR) analysis of rhNBD1 binding to P31-43 and P57-68 biotinylated peptides immobilized on SA sensor chip (c) and of increasing concentrations of P31-43 and P57-68 peptides 15 on rhNBD1 covalently bound to the CMS sensor chip (d).

e-f, Blue native PAGE western blotting of P31-43 and P57-68 biotinylated peptides in the presence of rhNBD1 (e), and of WT and double NBD1 mutants in the presence of biotinylated P31-43 (0. g, SPR analysis of 4QA peptide on rhNBD1 covalently bound to CMS sensor chip. h-i, treatment of Caco-2 cells with P31-43 or P31-43 modified in different Q or P positions. (h) Assessment in Ussing chambers of CFTR-dependent Cl⁻ secretion measured by forskolin-induced (Fsk) increase of the chloride current (Isc (μA/cm²); quantification of the peak CFTR Inhibitor 172 (CFTRinh172)-sensitive Isc (ΔIsc). (i) Assessment of iodide efflux by SPQ fluorescent probe upon stimulation with forskolin (Fsk) expressed as percentage of CFTR function in the presence or absence of peptide challenge. Caco-2 challenged with P57-68 or P31-43 or P31-43 modified in different Q (left) or P (right) positions.

FIG. 3: a, P31-43 induced modifications on NBD1 ATP binding site using the intrinsic W401 5 fluorescence. b, P31-43 effect on NBD1 ATPase activity.

FIG. 4: a, Incubation of Caco-2 cells with P57-68, P31-43 (a,b) or the P31-43/4QA mutant (4QA) (c), in the presence or absence of VX-770 (b). Incubation of Caco-2 cells for with 1 h with peptides (a-c) or P31-43 in the presence or absence of VX-770 (b). Immunoprecipitation of CFTR protein and immunblot with streptavidin.

FIG. 5: a, Immunoprecipitation of TG2 protein 15 in Caco-2 cells challenged with P57-68, PGAV or P31-43 and blotted with HRP-streptavidin. b, Immunoprecipitation of CFTR protein in Caco-2 cells challenged with P31-43 in the presence of absence of the TG2 inhibitor ZDON or TG2 RNA silencing and 20 blotted with anti-isopeptide glutamime-lysine and anti-TG2 antibodies. c, Recombinant TG2 and/or NBD1 incubated in vitro with P31-43 and solved in native page in the presence or absence of Ca2+. d, Immunoprecipitation of CFTR protein and immunblot with anti-CFTR antibodies or anti-TG2 antibodies. e, Immunoprecipitation of CFTR protein and immunoblot with anti-TG2 or anti-CFTR antibodies; effects of ZDON and BAPTA-AM. f, In situ detection of TG2 activity in Caco-2 cells pulsed with Ca2+ in the presence or absence of VX-770 or Z-DON. Activity assay by immunoblotting of the TG-catalyzed incorporation of 5-biotinamidopentylamine (BAP) and blotting with anti-biotin antibody. g, In situ TG2 activity assay, by immunostaining of the TG-catalysed incorporation of monodansylcadaverin, on Caco-2 cells pre-incubated with VX-770 or Z-DON and then pulsed with P31-43. Scale bar 50 μm. h, Immunoprecipitation of PKAr2a protein and immunoblot with isopeptide glutamime-lysine antibody in Caco-2 cells challenged with P57-68 or P31-43 in the absence or presence of VX-770. (i,j). Immunoblot of phospho-PKA (pPKA) protein (i) or PPARγ or phosphoERK 1/2 (pERK 1/2). k, Immunoblot of HSP70 in cell lysates.

FIG. 6: Immunoblot of BECN1 (a-b), hVps34 (c-d), UVRAG and Rab5 (e) in cell lysates from Caco-2 cells incubated with PGAV or P57-68 or P31-43 in the presence or absence of VX-770 or Vx-532 with/without CFTRinh172 (c) or 3-MA (e); f, Immunoblot of Rab5 and Rab7 proteins in endosomal protein fractions from Caco-2 cells challenged with P57-68 or P31-43 with/without VX-770; endosomal antigen-1 (EEA-1) as loading control. g, Immunoblot of purified protein from endosomal fractions with anti-TG2, anti-CFTR and anti-EEA-1 antibodies. EEA-1 was used as control of purification. h, Immunoprecipitation of CFTR protein and immunoblot with HRP-streptavidin of endosomal protein fractions. i, Caco-2 cells challenged with biotinylated P31-43 for 5 minutes at 37° C. Immunoblot of purified protein from membrane fractions with anti-TG2, anti-CFTR, anti-clathrin and anti-EEA-1 antibodies. EEA-1 and clathrin was used as control of purification of clathrin positive membrane fraction vesicles. Densitometric analysis of protein levels (bottom). Mean±SD of three independent experiments.

FIG. 7: Caco-2 cells challenged with P57-68 or P31-43 in the presence or absence of VX-770 or VX-532. a, Immunoblot of plasma membrane proteins with anti-SQSTM1\p62 and anti-flotillin antibodies. b, Immunoprecipitation of CFTR protein in membrane fractions and immunoblot with anti-ubiquitin or anti-CHIP or anti-CFTR antibodies. c,d, Immunoblot of purified plasma membrane proteins at 3, 6 and 24 h of peptide challenge with anti-CFTR and Flotillin antibodies (bottom) in the presence or absence of VX-770 or VX-532. Immunoblots representative of one of three independent experiments.

FIG. 8: a-b, NLRP3 expression (a) and caspase 1 cleavage (b) by immunoblotting with specific antibodies in Caco-2 cells challenged for 2 or 4 h in the presence or absence of VX-770. Immunoblot representative of one of three independent experiments; c, IL-15 production (quantified by specific ELISA) in CFTR-WT Caco-2 cells treated or not with P31-43 in presence or absence of VX-770; d, Immunoblotting with specific antibodies in Caco-2 cells challenged for 2 or 4 h in the presence or absence of VX-770. NF-kB p65 in cytoplasmic and nuclear extracts.

FIG. 9: a-g, BALB/c mice were fed for at least 3 generations with a gluten-free diet, orally challenged with vehicle or gliadin for 4 weeks (5 mg/daily for one week and then 5 mg/daily thrice a week for 3 weeks) in the presence or absence of intraperitoneal VX-770 administered 15 minutes prior gliadin challenge (n=10 mice per group of treatment). (a) Representative traces of CFTR dependent Cl-secretion measured by forskolin (Fsk)induced increase of chloride current (Isc (μA/cm2)) in small intestines mounted in Ussing chambers; quantification of the peak CFTR Inhibitor 172 (CFTRinh172)-sensitive Isc (ΔIsc) in tissue samples.

(b-d) Immunoblot with antibodies against CFTR (b), TG2 (c), pERK1\2 (d), and βactin as loading control, in whole lysates from small intestine homogenates. Immunoblot representative of one experiments. e, Plasma markers of intestinal permeability in mice. Plasma concentration of FITC-dextran 4000 (FITC-D4000) measured 1 h after gavage of a single dose of 600 mg FITCD4000 per kg body weight. Quantification of plasma concentration from n=10 mice per group of treatment expressed as mean±SD (***p<0.001, ANOVA, Bonferroni post test). f,g NOD mice (f) and NOD.scid AB0nullDQ8 mice (NOD-DQ8 mice) (g) orally challenged with vehicle or gliadin (for consecutive 4 weeks) in the presence or absence of intraperitoneal administration of VX-770 15 minutes prior gliadin challenge (n=10 mice per group of treatment). Assessment in Ussing chambers of CFTR-dependent Cl-secretion measured by forskolin-induced (Fsk) increase of the chloride current (Isc (μA/cm2); quantification of the peak CFTR Inhibitor 172 (CFTRinh172)-sensitive Isc (ΔIsc).

FIG. 10: BALB/c mice orally challenged with vehicle or gliadin (for consecutive 4 weeks) in the presence or absence of intraperitoneal administration of VX-770 or Genistein 15 minutes prior gliadin challenge (n=10 mice per group of treatment). α-b, NLRP3 expression (a) and caspase 1 cleavage (b) by immunoblotting with specific antibodies in small intestines from 3 mice. Immunoblot representative of three experiments. c, Confocal image staining with anti-NKG2D and nucleus (blue) in the small intestine. Scale bar, 20 μM. d, Transcript (left) or protein (by specific ELISA) (right) levels of IL-15, IL-21, IL-17A and IFN-γ. Mean±SD of pooled samples assayed in triplicates; *P<0.05, **P<0.01, ***P<0.001 vehicle vs gliadin; °P<0.05, °°P<0.01, °°°P<0.001 gliadin vs VX770+gliadin (ANOVA, Bonferroni post test). e, Transcript level of Rorc and Tbet from small intestine homogenates. Mean±SD of pooled samples assayed in triplicates; ****P<0.0001 gliadin vs VX770+gliadin (ANOVA, Bonferroni post test). f, Protein levels (by specific ELISA) of IL-10 and TGF-β from small intestine homogenates. Mean±SD of pooled samples assayed in triplicates; ****P<0.001 gliadin vs VX-770+gliadin (ANOVA, Bonferroni post test). g, Protein levels of IL-15, and IFN-γ from small intestine homogenates. Mean±SD of pooled samples assayed in triplicates; *P<0.05,***P<0.01 vs gliadin, P<0.01, vs Genistein treatment (ANOVA, Bonferroni post test). h, NOD mice challenged with gliadin for consecutive 4 weeks in the presence or absence of intraperitoneal administration of VX-770 15 minutes prior gliadin challenge (n=10 mice per group of treatment). IL-15, 30 IL-17A and IFN-γ protein levels from small intestine homogenates. Mean±SD of pooled samples assayed in triplicates; ***P<0.001 vs Gliadin, P<0.01 vs VX-770 treatment (ANOVA, Bonferroni post test). i, NOD.scid AB0nullDQ8 mice (NOD-DQ8 mice) orally challenged with vehicle or gliadin (for consecutive 4 weeks) in the presence or absence of intraperitoneal administration of VX-770 15 minutes prior gliadin challenge (n=10 mice per group of treatment). IFN-γ protein levels from small intestine homogenates. Mean±SD of pooled samples assayed in triplicates; **P<0.01 vs gliadin, P<0.05 vs VX-770 treatment (ANOVA, Bonferroni post test).

FIG. 11: Effects of VX-770 and genistein on IFN-γ (a, b) or IL-10 (c) release (ELISA) in culture supernatants by PBMC from 6 celiac patients (a-c) or 4 controls (a, b) cultured in the lower compartment of a bidimensional co-culture model upon 24 h challenge of confluent CaCo-2 cells in the upper compartment with PT-gliadin (a) or combination of P31-43 and P57-68 (b) in presence or absence of VX-770 (a-c) or genistein (b). ***p<0.001, PT-gliadin vs medium; °°°p<0.001, PT-gliadin vs PT-gliadin+VX-770 (n=4); ***P<0.001, P57-68 vs P31-43/P57-68 combination (n=6); °°°p<0.001, P57-68/P31-43 combination vs VX-770 treatment (n=3); ## p<0.01 P57-68\P31-43 combination vs genistein treatment (n=3) (ANOVA, Bonferroni post hoc test). (c) IL-10 release (ELISA) by PBMC from 4 celiac patients cultured as in (b). ****p<0.0001, P57-68/P31-43 combination vs P57-68/P31-43+VX-770 treatment (n=3).

### REFERENCES

Barone, M. V., et al. Gliadin peptides as triggers of the proliferative and stress/innate immune response of the celiac small intestinal mucosa. Int J Mol Sci. 15, 20518-37 (2014).
Bouziat, R., et al. Reovirus infection triggers inflammatory responses to dietary antigens and development of celiac disease. Science 356, 44-50 (2017).
Cerf-Bensussan, N. et al. Coeliac disease & gluten sensitivity: Epithelial stress enters the dance in coeliac disease. Nat Rev Gastroenterol Hepatol. 12, 491-7 (2015).
Chin, S., et al. Current insights into the role of PKA phosphorylation in CFTR channel activity and the pharmacological rescue of cystic fibrosis disease-causing mutants. Cell Mol Life Sci. 74, 57-66 (2017).
Chen, R., Li, L., et al. ZDOCK: An Initial-stage Protein Docking Algorithm. Proteins 52, 80-87 (2003).
De Lisle, R. C., et al. The cystic fibrosis intestine.
Cold Spring Harb Perspect Med. 1,3:a009753 (2013).
De Stefano, D., et al. Restoration of CFTR function in patients with cystic fibrosis carrying the F508del-CFTR mutation. Autophagy 10, 2053-74(2014).
Eugster, P. J. et al. Production and characterization of two major Aspergillus oryzae secreted prolyl endopeptidases able to efficiently digest proline-rich peptides of gliadin. Microbiology. 161, 2277-88 (2015).
Galipeau, H. J, et al. Sensitization to gliadin induces moderate enteropathy and insulitis in nonobese diabetic-DQ8 mice. J Immunol. 187, 4338-46 (2011).
Goel, T. et al. Epitope-specific immunotherapy targeting CD4-positive T cells in coeliac disease: two randomised, double-blind, placebo-controlled phase 1 studies. Lancet Gastroenterol Hepatol. 7, 479-493 (2017).
Kroemer, G., et al. Autophagy and the integrated stress response. Mol Cell 40, 280-293 (2010).
Jabri, B. et al. IL-15 functions as a danger signal to regulate tissue-resident T cells and tissue 20 destruction. Nat Rev Immunol. 15, 771-83 (2015).
Jih, K. Y et al. Vx-770 potentiates CFTR function by promoting decoupling between the gating cycle and ATP hydrolysis cycle. Proc Natl Acad Sci USA. 110, 4404-9 25 (2013).
Luciani, A., et al. SUMOylation of tissue transglutaminase as link between oxidative stress and 35 inflammation. J Immunol. 183, 2775-8 (2009).
Luciani, A., et al. Lysosomal accumulation of gliadin p31-43 peptide induces oxidative stress and tissue transglutaminase-mediated PPARgamma downregulation in intestinal epithelial cells and coeliac mucosa. Gut. 59, 311-9 (2010).
Luciani, A., et al. Defective CFTR induces aggresome formation and lung inflammation in cystic fibrosis through ROS-mediated autophagy inhibition. Nat Cell Biol. 12, 863-75 (2010)
Luciani, A., et al. Targeting autophagy as a novel strategy for facilitating the therapeutic action of potentiators on ΔF508 cystic fibrosis transmembrane conductance regulator. Autophagy. 8, 1657-72 (2012).
Lukacs, G. L., et al. Constitutive internalization of cystic fibrosis transmembrane conductance regulator occurs via clathrin-dependent endocytosis and is regulated by protein phosphorylation. Biochem J. 328, 353-61(1997).
Maiuri, L., et al. Association between innate response to gliadin and activation of pathogenic T cells in coeliac disease. Lancet. 362, 30-7 (2003).
Maiuri, L., et al. Tissue transglutaminase activation modulates inflammation in cystic fibrosis via PPARgamma 20 down-regulation. J Immunol. 180, 7697-705 (2008).
Maurano, F., et al. Small intestinal enteropathy in non-obese diabetic mice fed a diet containing wheat. Diabetologia. 48, 931-7 (2005).
Meresse, B., et al. Celiac disease: from oral tolerance to intestinal inflammation, autoimmunity and lymphomagenesis. Mucosal Immunol. 2, 8-23 (2009).
Meresse, B., et al. Celiac disease: an immunological jigsaw. Immunity 36, 907-919 (2012).
Moon, S. H., et al. Sensitization to and Challenge with Gliadin Induce Pancreatitis and Extrapancreatic Inflammation in HLA-DQ8 Mice: An Animal Model of Type 1 Autoimmune Pancreatitis. Gut Liver. 10, 842-50 (2016).
Palova-Jelinkova, L., et al. Pepsin digest of wheat gliadin fraction increases production of IL-1β via TLR4/MyD88/TRIF/MAPK/NF-κB signaling pathway and an NLRP3 inflammasome activation. PLoS One. 8, e62426 (2013).
Papista, C., et al. Gluten induces coeliac-like disease in sensitised mice involving IgA, CD71 and transglutaminase 2 interactions that are prevented by 10 probiotics. Lab Invest. 92, 625-35 (2012).
Rauhavirta, T., et al. Are transglutaminase 2 inhibitors able to reduce gliadin-induced toxicity related to celiac disease? A proof-of-concept study. J Clin Immunol. 33, 134-42 (2013).
Romani, L., et al. Thymosin al represents a potential potent single-molecule-based therapy for cystic fibrosis. Nat Med. 23, 590-600 (2017).
Sheppard, D. N. et al. Structure and function of the CFTR chloride channel. Physiol Rev. 79(1 Suppl):523-45 25 (1999).
Siegel, M. et al. Transglutaminase 2 inhibitors and their therapeutic role in disease states. Pharmacol Ther. 115, 232-45 (2007).
Sollid, L. M. et al. Celiac disease and transglutaminase 2: a model for posttranslational modification of antigens and HLA association in the pathogenesis of autoimmune disorders. Curr Opin Immunol. 23, 732-8 (2011).
Sollid, L. M et al. Triggers and drivers of autoimmunity: lessons from coeliac disease. Nat Rev Immunol. 13, 294-302 (2013).
Tosco A, et al. A novel treatment of cystic fibrosis acting on-target: cysteamine plus epigallocatechin gallate for the autophagy-dependent rescue of class Ilmutated CFTR. Cell Death Differ. 23, 1380-93 (2016).
Villella, V. R., et al. Disease-relevant proteostasis regulation of cystic fibrosis transmembrane conductance regulator. Cell Death Differ. 20, 1101-15 (2013).
Verkman, A. S. et al. Chloride channels as drug 15 targets. Nat Rev Drug Discov 8, 153-171 (2009).
Volynets, V., et al. Assessment of the Intestinal Barrier with Five Different Permeability Tests in Healthy C57BL/6J and BALB/cJ Mice. Dig Dis Sci. 61, 20 737-46 (2016).
Zimmer, K. P., et al. Endocytotic segregation of gliadin peptide 31-49 in enterocytes. Gut. 59, 300-10 (2010).

1. A method of treating and/or preventing at least one intestinal and/or extra-intestinal condition of gluten sensitivity selected from celiac disease, non-celiac gluten sensitivity, potential celiac disease, refractory celiac disease, type diabetes, autoimmune thyroiditis and irritable bowel disease with a CFTR channel activator, said method comprising
administering a pharmaceutically effective amount of said CFTR channel activator to a subject in need thereof.

2. The method according to claim 1, wherein said activator is a CFTR potentiator or amplifier.

3. The method CFTR channel activator for use according to claim 1, wherein said activator is a CFTR potentiator.

4. The method according to claim 3, wherein the CFTR potentiator is selected from N-(2,4di-tert-butyl-5-hydroxyphenyl)-4-oxo-1,4dihydroquinoline-3-carboxamide, 2-[(2-1H-indol-3-ylacetyl)-methyl-amino]-N-(4-isopropyl-phenyl)-2phenyl-acetamide (PG01,P2), 4-Methyl-2-(5-phenyl-1Hpyrazol-3-yl)-phenol, 6-(Ethyl-phenyl-sulfonyl)-4oxo-1,4-dihydro-quinoline-3-carboxylic acid 2-methoxy-benzylamide, 1-(3-chlorophenyl)5-trifluoromethyl-3-hydrobenzimidazol-2-one, 2-(2-Chloro-benzoylamino)-4,5,6,7-tetrahydro-benzo[b]thiophene-3-carboxylic acid amide, 5,7,Dihydroxy-3-(4-hydroxy-phenyl)-chroman-4-one, 1-(5-Chloro-2-hydroxy-phenyl)-5-trifluoromethyl-1,3-dihydro-indol-2-one), 4-(4-Oxo4H-benzo[h]chromen-2-yl)-pyridinium; bisulfate, 3-But-3-ynyl-5-methoxy-1-phenyl-1H-pyrazole-4-carbaldehyde, 3-(2-Benzyloxy-phenyl)-5-chloromethyl-isoxazole, 5-Nitro-2-(3-Phenylpropylamino) Benzoate, 4,6,4′-trimethylangelicin, or mixtures thereof.

5. The method according to claim 4, wherein the CFTR potentiator is selected from N-(2,4di-tert-butyl-5-hydroxyphenyl)-4-oxo-1,4dihydroquinoline-3-carboxamide, 5,7,Dihydroxy-3-(4-hydroxy-phenyl)-chroman-4-one, 4-Methyl-2-(5-phenyl-1H-pyrazol-3-yl)-phenol or mixtures thereof.

6. The method according to claim 2, wherein the CFTR amplifier is PTI-428.

7. The method according to claim 1, wherein the functional gastrointestinal disorder is the irritable bowel syndrome (IBS).

8. A method of treating or preventing celiac disease, non-celiac gluten sensitivity, potential celiac disease, refractory celiac disease, type diabetes, autoimmune thyroiditis with a pharmaceutical composition comprising the CFTR channel activator according to claim 1 and at least one pharmaceutically acceptable excipient, said method comprising
administering a pharmaceutically effective amount of said pharmaceutical composition to a subject in need thereof.

9. The method according to claim 8, wherein said pharmaceutical composition is in form of small intestine-specific drug oral administration forms.


SEQUENCE LISTING (4 sequences)

SEQ ID NO: 1 - protein, 13 aa, Artificial Sequence
  P31-43  sequence of alpha-gliadin
LGQQQPFPPQQPY

SEQ ID NO: 2 - protein, 12 aa, Artificial Sequence
  P57-68 sequence of alpha-gliadin
QLQPFPQPQLPY

SEQ ID NO: 3 - protein, 12 aa, Artificial Sequence
  control scrambled peptide
GAVAAVGVVAGA

SEQ ID NO: 4 - protein, 13 aa, Artificial Sequence
  P31-43 sequence of alpha gliadin with glutamine residues at positions 4, 5, 10 and 11 substituted with alanines
LGQAAPFPPAAPY